(19) United States
(12) Patent Application Publication
(10) Pub. No.: US 2025/0215413 A1
(43) Pub. Date: Jul. 3, 2025

(54) BIO-ENGINEERED ENZYMES AND USES THEREOF

(71) Applicant: The Johns Hopkins University, Baltimore, MD (US)

(72) Inventors: Taekjip Ha, Baltimore, MD (US); Momcilo Gavrilov, Baltimore, MD (US)

(21) Appl. No.: 18/833,584

(22) PCT Filed: Jan. 30, 2023
(86) PCT No.: PCT/US23/61592

§ 371 Date: Jul. 26, 2024

Related U.S. Application Data
(60) Provisional application No. 63/304,189, filed on Jan. 28, 2022.

(51) Int. Cl. C12N 9/90; C12Q 1/533; C12Q 1/6844

(52) U.S. Cl. CPC C12N 9/90; C12Q 1/533; C12Q 1/6844

(57) ABSTRACT

Engineered enzymes for amplification of nucleic acid sequences which function at constant temperatures thereby eliminating heating and cooling cycles associated with traditional polymerase chain reaction (PCR) are disclosed. Specifically, novel, genetically engineered PcrA helicase enzymes are utilized in conjunction with amplification components to provide a method for amplification of nucleic acid sequence by incubating at a substantially isothermal temperature i) a composition comprising a target sequence, ii) primers and iii) said engineered PcrA helicase, a single stranded binding protein (SSB), a polymerase, and/or a thermostable pyrophosphatase (PPase) buffer, or combinations thereof.

### CROSS-REFERENCES TO RELATED APPLICATION

This application claims the benefit of priority of U.S. Provisional Application No. 63/304,189 filed on Jan. 28, 2022, which is incorporated herein by reference in its entirety and for all purposes.

### STATEMENT REGARDING FEDERALLY SPONSORED RESEARCH

This invention was made with government support under grant GM122569 awarded by the National Institutes of Health. The government has certain rights in the invention.

### FIELD

Enzymes are provided for isothermal amplification of nucleic acid sequences, without the need for heating and cooling cycles associated with traditional polymerase chain reaction (PCR).

### BACKGROUND

DNA and RNA amplification is an essential method involved in rapid molecular diagnostics, gene manipulations, and genetic analyses including the detection of bacteria, viruses, and diagnosis of genetic disorders. The most widely used method for DNA amplification is polymerase chain reaction (PCR), implemented through thermal cycling. Promising next generation methods include enzymatic isothermal amplification; however, existing isothermal methods are limited to producing only short amplicons, or complex heterogeneous mixed and branched products, and often involve using multiple sets of complicated pairs of primers (Y. Zhao, F. Chen, Q. Li, L. Wang and C. Fan, “Isothermal Amplification of Nucleic Acids,” Chemical Reviews, vol. 115, p. 12491-12545, 11 2015). As a result, no existing isothermal method can approach the versatility of thermal cycler-based PCR.

### SUMMARY

Provided herein is a polymerase chain reaction (PCR) which eliminates thermal cycling while retaining many of the desired traditional PCR features, termed herein as SHARP (SSB-Helicase Assisted Rapid PCR). Compositions include a novel helicase and an SSB (Single-Stranded Binding protein) for effective strand separation at constant temperature, thus avoiding the need for periodic heating. SHARP uses the same starting set of primers and template DNA as PCR does, carries out the reaction at a constant temperature, and outputs the same amplicon as PCR with lengths of up to 6000 base pairs, a feature no existing isothermal amplification method can match.

Accordingly, in certain embodiments, a bacterial derived helicase comprises a PcrA helicase having at least two or more mutations. In certain embodiments, the PcrA helicase comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 90%, 91%, 92%, 93%, 94%, 95%, 96%, 97%, 98%, or 99%, sequence identity to SEQ ID NO: 1. In certain embodiments, the PcrA helicase comprises at least a 95%, 96%, 97%, 98%, or 99%, sequence identity to SEQ ID NO: 1. In certain embodiments, the PcrA helicase comprises SEQ ID NO: 1. In certain embodiments, the PcrA helicase is derived from Geobacillus. In certain embodiments, the Geobacillus is Geobacillus stearothermophilus.

In certain embodiments, a vector encodes a PcrA helicase, wherein the PcrA helicase comprises a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 90%, 91%, 92%, 93%, 94%, 95%, 96%, 97%, 98%, or 99%, sequence identity to SEQ ID NO: 1. In certain embodiments, the PcrA helicase comprises at least a 95%, 96%, 97%, 98%, or 99%, sequence identity to SEQ ID NO: 1. In certain embodiments, the PcrA helicase comprises SEQ ID NO: 1. In certain embodiments, the PcrA helicase is derived from Geobacillus stearothermophilus.

In certain embodiments, a vector comprises a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, a vector comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, a vector comprises at least an 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, a vector comprises at least a 90%, 91%, 92%, 93%, 94%, 95%, 96%, 97%, 98%, or 99%, sequence identity to SEQ ID NO: 2. In certain embodiments, a vector comprises SEQ ID NO: 2.

In certain embodiments, a nucleic acid sequence comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 3. In certain embodiments, the nucleic acid sequence comprises at least a 75%, 80%, 85%, 86%, 87%, 88%, 89%, 90%, 91%, 92%, 93%, 94%, 95%, 96%, 97%, 98%, or 99% sequence identity to SEQ ID NO: 3. In certain embodiments, the nucleic acid sequence comprises at least a 90%, 91%, 92%, 93%, 94%, 95%, 96%, 97%, 98%, or 99% sequence identity to SEQ ID NO: 3. In certain embodiments, a nucleic acid sequence comprises SEQ ID NO: 3. In certain embodiments, SEQ ID NO: 3 encodes an amino acid comprising amino acid substitutions at positions H93A, C96A, N187C, C247A, L384V and L409C.

In certain embodiments, an engineered helicase comprises a PcrA helicase wherein said PcrA helicase comprises one or more amino acid mutations. In certain embodiments, the PcrA helicase is derived from Geobacillus stearothermophilus. In certain embodiments, the PcrA helicase comprises mutations at amino positions 93, 96, 187, 247, 384 and 409 of wild type PcrA helicase.

In certain embodiments, a method of amplifying nucleic acids comprises: mixing a first composition with one or more primers, a target nucleic acid sequence, and a second composition. In certain embodiments, the mixed compositions are incubated for about 20-120 minutes preferably at a substantially constant temperature and preferably in a temperature range of less than 100° C. such as between 37° C. to 60° C., 70° C., 80° C. or 90°° C. In certain embodiments, the mixed composition is incubated up to about 10, 20, 30 or 40 minutes at a substantially constant temperature (substantially contant temperature being e.g. within 1° C., 2° C., 3° C., 4° C. or 5° C., 6° C., 7° C. 8° C., 9° C. or 10° C. of a specified temperature), such as, for example, of up to about 45° C., 50° C., 55° C., 60° C., 65° C., 70° C., 75° C., 80° C., 85° C. or 90° C. In certain embodiments, the mixed composition is incubated at a constant temperature or substantially constant temperature, such as, for example, of about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C.

In certain embodiments, the first composition comprises a nucleic acid dye, deoxyribonucleotide triphosphates (dNTPs), adenosine triphosphate (ATP) or combinations thereof. In certain embodiments, the second composition comprises: an engineered PcrA helicase, a single stranded binding protein (SSB), a polymerase, a thermostable pyrophosphatase (PPase) buffer or combinations thereof. In certain embodiments, the engineered PcrA helicase comprises SEQ ID NO: 1.

In certain embodiments, the first and second compositions further comprise: water, buffers or the combination thereof. In certain embodiments, the polymerase comprises: DNA polymerase (DNAP) I, DNAP II, DNAP III, DNAP IV, DNAP V, Klenow fragment, reverse transcriptases, Exo-polymerase, high-fidelity polymerases, Taq polymerases, bacteriophage polymerases, Pyrococcus furiosus Vc1 (Pfu) polymerase, Hot Start DNA Polymerase, engineered polymerases, engineered reverse transcriptases or eukaryotic polymerases. In certain embodiments, the polymerase is a Geobacillus stearothermophilus DNA polymerase I, Bacillus subtilis DNA polymerase I, Klenow fragment, Exo-polymerase, Bst, Bst 2.0 or Bst 3.0. In certain embodiments, the Geobacillus stearothermophilus DNA polymerase I is Bst polymerase or Bst-large fragment polymerase (Bst-LF). In certain embodiments, the reverse transcriptase comprises murine leukemia virus (MMLV) reverse transcriptase or Reverse Transcription Xenopolymerase (RTX).

In certain embodiments, a kit comprises an engineered PcrA helicase, a single stranded binding protein (SSB), a polymerase, a thermostable pyrophosphatase (PPase). In certain embodiments, the engineered PcrA helicase comprises SEQ ID NO: 1. In certain embodiments, the polymerase comprises: DNA polymerase (DNAP) I, DNAP II, DNAP III, DNAP IV, DNAP V, Klenow fragment, reverse transcriptases, Exo-polymerase, high-fidelity polymerases, Taq polymerases, bacteriophage polymerases, Pyrococcus furiosus Vc1 (Pfu) polymerase, Hot Start DNA Polymerase engineered polymerases, engineered reverse transcriptases or eukaryotic polymerases. In certain embodiments, the polymerase is a Geobacillus stearothermophilus DNA polymerase I, Bacillus subtilis DNA polymerase I, Klenow fragment, Exo-polymerase, Bst, Bst 2.0 or Bst 3.0. In certain embodiments, the Geobacillus stearothermophilus DNA polymerase I is Bst polymerase or Bst-large fragment polymerase (Bst-LF). In certain embodiments, the reverse transcriptase comprises murine leukemia virus (MMLV) reverse transcriptase or Reverse Transcription Xenopolymerase (RTX).

In certain embodiments, the kit further comprises buffers, deoxyribonucleotide triphosphates (dNTPs), nucleoside triphosphates (NTPs), a detectable nucleic acid label, water, dithiothreitol (DTT) or combinations thereof. In certain embodiments, the detectable nucleic acid label comprises a fluorescent dye.

In certain embodiments, a kit comprises a first composition and a second composition. In certain embodiments, the first composition comprises: a nucleic acid dye, deoxyribonucleotide triphosphates (dNTPs), adenosine triphosphate (ATP) or combinations thereof. In certain embodiments, the second composition comprises: an engineered PcrA helicase, a single stranded binding protein (SSB), a polymerase, a thermostable pyrophosphatase (PPase) buffer or combinations thereof. In certain embodiments, the first and second compositions further comprise; water, buffers or the combination thereof. In certain embodiments, the polymerase comprises: DNA polymerase (DNAP) I, DNAP II, DNAP III, DNAP IV, DNAP V, Klenow fragment, reverse transcriptases, Exo-polymerase, high-fidelity polymerases, Taq polymerases, bacteriophage polymerases, Pyrococcus furiosus Vc1 (Pfu) polymerase, Hot Start DNA Polymerase, engineered polymerases, engineered reverse transcriptases or eukaryotic polymerases. In certain embodiments, the polymerase is a Geobacillus stearothermophilus DNA polymerase I, Bacillus subtilis DNA polymerase I, Klenow fragment, Exo-polymerase, Bst, Bst 2.0 or Bst 3.0. In certain embodiments, the Geobacillus stearothermophilus DNA polymerase I is Bst polymerase or Bst-large fragment polymerase (Bst-LF). In certain embodiments, the reverse transcriptase comprises murine leukemia virus (MMLV) reverse transcriptase or Reverse Transcription Xenopolymerase (RTX). In certain embodiments, the first composition is contained in a first vial and the second composition is contained within a second vial.

In certain embodiments, a method of amplifying nucleic acids comprises incubating a composition comprising a target sequence with primers and an engineered PcrA helicase, a single stranded binding protein (SSB), a polymerase, a thermostable pyrophosphatase (PPase) buffer or combinations thereof, at a substantially constant temperature and preferably in a temperature range of less than 100° C. such as between 37° C. to 90° C.

In certain embodiments, the composition is incubated at a temperature (preferably isothermal temperature) of about 45° C. or less, or 40° C. or less, or 38° C. or less such as about 30° C., 31° C., 32° C., 35° C., 36° C., 37° C., 38° C., 39° C. or 40° C.

In certain embodiments, the composition is incubated for about 5 to 120 minutes, or more typically 10 or 20 minutes to about 60 minutes, including up to 10, 20, 30, 40, 50, 60, 70, 80, 90, 100, 110, 120, 130, 140, 150, 160, 170 or 180 minutes, at isothermal conditions.

In certain embodiments, the mixed composition is incubated at a constant temperature, such as, for example, of up to or about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C.

In certain embodiments, the engineered PcrA helicase comprises at least a 95% sequence identity to SEQ ID NO: 1. In certain embodiments, the engineered PcrA helicase comprises SEQ ID NO: 1. In certain embodiments, the target nucleic acid sequence is an RNA, DNA or the combination thereof.

In one preferred aspect, a method of amplifying nucleic acids comprising: incubating at a substantially isothermal temperature i) a composition comprising a target sequence, ii) primers and iii) an engineered PcrA helicase, a single stranded binding protein (SSB), a polymerase, and/or a thermostable pyrophosphatase (PPase) buffer, or combinations thereof.

In a further provided aspect, a method of amplifying nucleic acids is provided comprising: incubating in the absence of thermal cycling i) a composition comprising a target sequence, iii) primers and iii) an engineered PcrA helicase, a single stranded binding protein (SSB), a polymerase, and/or a thermostable pyrophosphatase (PPase) buffer, or combinations thereof.

In such methods, the composition may be suitably incubated at about 40° C. to about 80° C., including at about 45° C. or less, or about 40° C. or less. The composition is suitably incubated for a sufficient time, such as 5 or 10 minutes to 60, 120 or 180 minutes or more.

In certain preferred aspects of such methods, suitably the engineered PcrA helicase comprises at least an 80%, 85% or 90% sequence identity to SEQ ID NO: 1. In certain aspects, the engineered PcrA helicase comprises at least a 95% sequence identity to SEQ ID NO: 1. In certain aspects, the engineered PcrA helicase comprises SEQ ID NO: 1.

In preferred methods, assays and systems, the amplification is sensitive, specific and/or can generate greater (e.g. 1.05, 1.1, 1.2, 1.4, 1.6, 1.8, 2, 3, 4 or 5 times greater) than or equal to kilo-base pair amplified product length as compared to PCR methods or reference PCR assay. Such sensitivity can be determined by one or more assays, e.g. wherein the one or more assays comprise quantitative or real-time PCR (qPCR) or by detecting a cycle threshold value, suitably wherein the cycle threshold value is the number of cycles after which the fluorescence of a PCR product can be detected above a background. See M. Gavrilov et al., Nature Communications (2022) 13:6312, incorporated herein by reference in its entirety.

In certain embodiments, a method of screening for cancer comprises the method or kits embodied herein. Cancer includes benign and malignant cancers as well as dormant tumors or micrometastatses. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In certain embodiments, a method of detecting infectious agents or sepsis comprising the method or kits embodied herein. The infectious agents comprise, for example, viruses, bacteria, fungi, prions, or parasites.

In certain embodiments, a method of diagnosing a disease or disorder in a subject comprising the method or kits embodied herein. The diseases or disorders comprise, for example: autoimmune diseases, cancer, inflammatory diseases, neurological diseases or disorders, neuroinflammatory diseases or disorders, cardiovascular disease, diabetes.

### Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “about” in the context of a numerical value or range means±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

The term “amino acid” as used herein refers to naturally occurring and synthetic α, β, γ, and δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. When the term amino acid is used, it is considered to be a specific and independent disclosure of each of the esters of α, β, γ, and δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “dNTP” refers to deoxyribonucleoside triphosphates. Non-limiting examples of such dNTPs are dATP, dGTP, dCTP, dTTP, dUTP, which may also be present in the form of labelled derivatives, for instance comprising a fluorescence label, a radioactive label, a biotin label. dNTPs with modified nucleotide bases are also encompassed, wherein the nucleotide bases are for example hypoxanthine, xanthine, 7-methylguanine, inosine, xanthinosine, 7-methylguanosine, 5,6-dihydrouracil, 5-methylcytosine, pseudouridine, dihydrouridine, 5-methylcytidine. Furthermore, ddNTPS of the above-described molecules are encompassed in the present invention.

The term “helicase” refers here to any enzyme capable of unwinding a double stranded nucleic acid enzymatically. For example, helicases are enzymes that are found in all organisms and in all processes that involve nucleic acid such as replication, recombination, repair, transcription, translation and RNA splicing. (Kornberg and Baker, DNA Replication, W. H. Freeman and Company (2^{nd} ed. (1992)), especially chapter 11).

“Isothermal amplification” refers to amplification which occurs at a single or constant temperature. This may not include the single brief time period (less than 15 minutes) at the initiation of amplification which may be conducted at the same temperature as the amplification procedure or at a higher temperature. Depending on the source of enzymes that are used for HDA, the reaction can be performed at low temperatures (<50° C.) e.g., at least or about 30, 31, 32, 33, 34, 35, 36, 37, 38, 39, 40, 41, 42, 43, 44, 45, 46, 47, 48, 49 degrees Celsius; or at high temperatures (>50° C.), e.g., at least or about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115 or more degrees Celsius.

As used herein, a “natural amino acid” refers to the twenty genetically encoded alpha-amino acids. See, e.g., Biochemistry by L. Stryer, 3^{rd} ed. 1988, Freeman and Company, New York for structures of the twenty natural amino acids.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50amino acids long, or at least 70 amino acids long.

As used herein, an “unnatural amino acid,” “non-natural”, “modified amino acid” or “chemically modified amino acid” refers to any amino acid, modified amino acid, or amino acid analogue other than the twenty genetically encoded alpha-amino acids. Unnatural amino acids have side chain groups that distinguish them from the natural amino acids, although unnatural amino acids can be naturally occurring compounds other than the twenty proteinogenic alpha-amino acids. In addition to side chain groups that distinguish them from the natural amino acids, unnatural amino acids may have an extended backbone such as beta-amino acids.

Non-limiting examples of non-natural amino acids include selenocysteine, pyrrolysine, homocysteine, an O-methyl-L-tyrosine, an L-3-(2-naphthyl) alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline. In an embodiment of the helicases described herein, one or more amino acids of the helicase are substituted with one or more unnatural amino acids and/or one or more natural amino acids.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

The terms “subject”, “patient” or “individual” are used interchangeably herein, and refers to a mammalian subject to be treated, with human patients being preferred. In some cases, the methods of the disclosure find use in experimental animals, in veterinary application, and in the development of animal models for disease, including, but not limited to, rodents including mice, rats, and hamsters; and primates. Patients in need of therapy comprise those at risk of developing a certain condition, disease or disorder (e.g. due to genetic, environmental or physical attributes, such as for example, obesity). Patients in need of therapy also include those afflicted with a condition, disease or disorder. The diseases or disorders comprise, for example: autoimmune diseases, cancer, inflammatory diseases, neurological diseases or disorders, neuroinflammatory diseases or disorders, cardiovascular disease, obesity, diseases or disorders caused by infectious agents such as, for example, viruses, bacteria, fungi, prions, or parasites.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

Any compositions or methods provided herein can be combined with one or more of any of the other compositions and methods provided herein.

### BRIEF DESCRIPTION OF THE DRAWINGS

FIGS. 1A and 1B are schematic representations showing a comparison of PCR and SHARP. FIG. 1A: PCR alternates the temperature between high (≈98° C.) to melt the DNA duplex and low (45 to 65° C.) to replicate the complementary strand by a DNAP. FIG. 1B: SHARP uses PcrA M6 helicase and SSB to open the duplex and enable primer binding, followed by Bst-LF DNAP to replicate the strand.

FIGS. 2A-2F are a series of plots, blots and a schematic representation showing a comparison of PCR and SHARP amplification product. FIG. 2A: 2019 coronavirus nCOV-2 N protein sequence between M13 primers. FIG. 2B: PCR product and detection limit for the template-primer set in FIG. 2A. FIG. 2C: Kinetics of the SHARP reaction for the template primer set in FIG. 2A. FIG. 2D: SHARP product and detection limit enables the direct comparison with PCR. FIG. 2E: SHARP doubling time estimate. Detection time is the intercept of the intensity curve with the threshold in FIG. 2C. FIG. 2F: SHARP essential components. In the absence of ATP, SSB, or PcrA M6 the reaction fails.

FIGS. 3A-3F are a series of blots and graphs showing other SHARP products. FIG. 3A: 3 kbp SHARP amplicon. FIG. 3B: 6 kbp SHARP amplicon. FIG. 3C: 200 bp SHAR amplicon using lambda DNA as a template. FIG. 3D: SHARP doubling time estimate for 200 bp product. FIG. 3E: Detection limit versus the amplicon length. For short amplicons, SHARP can detect 1 molecule in a test tube. FIG. 3F: Doubling time shows that short amplicons replicate at the highest speed.

FIGS. 4A-4E are a series of graphs, blots and a schematic representation demonstrating the transformation and properties of the SHARP plasmid. FIG. 4A: Transforming SHARP-made plasmid to E. coli. FIG. 4B: SHARP amplifies sequence prone to forming secondary structure, CAG repeats. FIG. 4C: SHARP kinetics curves at different temperatures, inset shows detection times. FIG. 4D: SHARP product at different temperatures. FIG. 4E: SHARP at 37° C. with Bsu and Klenow Exo-DNAp at variable SSB concentration.

FIGS. 5A-5I are a series of graphs and a schematic representation demonstrating the bulk unwinding activity of different DNA helicases at 37° C. Each helicase is at different concentration, while all other conditions are fixed, see Methods. FIG. 5A: Unwinding reaction of FRET-pair labelled DNA construct. FIG. 5B: Rep-X superhelicase shows highest unwinding activity even at low concentration of 10 nM. C) E coli UvrD helicase at 34 nM shows relatively high initial unwinding activity, but the reaction terminates quickly resulting in the DNA reannealing. FIG. 5D: Thermostable Tte UvrD helicase at 40 nM shows no unwinding activity at 37° C. FIG. 5E: Wild-type PcrA expressed with 6xHis tag shows low unwinding activity at 600 nM. FIG. 5F: Wild-type PcrA without 6xHis tag shows low unwinding activity at 600 nM. FIG. 5G: PcrA M5 mutant at 400 nM shows high unwinding activity. FIG. 5H: PcrA M5 mutant at 40 nM. FIG. 5I: PcrA M6 shows higher unwinding activity than M5 even at 40 nM concentration. SHARP uses about 40 nM of PcrA M6.

FIGS. 6A-6D are a series of schematic representations and graphs showing the structure and characterization of PcrA M6 mutant for unwinding long DNA. FIG. 6A: Structure of wild-type PcrA and location of point mutations H93A, C96A, N187C, C247A, L384V, L409C. FIG. 6B: Unwinding of λDNA occurred in the presence of SSB and ATP. FIG. 6C: Unwinding of different DNA substrates. FIG. 6D: Time needed unwind different DNA substrates by PcrA M6.

FIGS. 7A-7L depict bulk unwinding activity of different DNA helicases at 37° C. Each helicase is at different concentration, while all other conditions are fixed. FIG. 7A: Unwinding reaction of FRET-pair labelled DNA construct. FIG. 7B: Rep-X superhelicase shows the highest unwinding activity even at a low concentration of 10 nM. FIG. 7C: E. coli UvrD helicase at 34 nM shows high initial unwinding activity, but the reaction terminates quickly resulting in the DNA reannealing likely due to ATP depletion. FIG. 7D: Thermostable Tte UvrD helicase at 40 nM shows very low unwinding activity at 37° C. FIG. 7E: Wild-type PcrA expressed with 6xHis tag shows low unwinding activity at 600 nM. FIG. 7F: Wild-type PcrA without 6xHis tag shows low unwinding activity at 600 nM. FIG. 7G: PcrA M5 mutant at 400 nM shows high unwinding activity. FIG. 7H: PcrA M5 mutant at 40 nM. FIG. 7I: PcrA M6 shows higher unwinding activity than M5 even at 40 nM concentration. SHARP uses nearly 40 nM of PcrA M6. FIG. 7J: Unwinding rates obtained from the fit to FRET efficiency in FIGS. 7B-7I. FIG. 7K: Unwinding activity relative to Rep-X is obtained by dividing unwinding rates in FIG. 7J by the concentration and normalizing. Inset shows relative activity on log10 axis. Data in FIG. 7J and FIG. 7K are presented as mean values±standard error obtained from the fit. Source data and fitting parameters are provided as a Source Data file.

FIGS. 8A-8C show single-molecule nanopore assay measuring the unwinding speed of PcrA M5 and PcrA M6 helicase. FIG. 8A: The Single-molecule Picometer Resolution Nanopore Tweezers (SPRNT) setup consists of two separate chambers connected with a nanopore. A helicase unwinds and drives the periodic DNA sequence through the pore and the electric current signal is recorded. By measuring the period of the signal repeat, where each period corresponds to 103 base pair sequence, we can infer the helicase speed. FIG. 8B: PcrA M5 shows around 3 repeats in 8 sec, hence we estimate the speed to 41 bp/s. FIG. 8C: PcrA M5 shows more than 10 repeats in just 8 s; hence we estimate its speed to 134 bp/s.

FIG. 9: SHARP can amplify RNA targets. Using MMLV reverse transcriptase and CDC N2 primers, we amplified the 67 bp region in nCOV-2 RNA sequence at different temperatures. The highest yield was obtained at 39.4 degrees Celsius.

FIGS. 10A-10C SHARP product can easily be detected with the lateral flow device for the use in portable, point-of-care, point-of-need, and home molecular diagnostics. FIG. 10A: A SHARP with biotin labeled primer and DIG-labeled dNTPs. FIG. 10B: SHARP is detected with the lateral flow device. FIG. 10C: In case no labeled dNTPs are used, the product does not show on the lateral flow devoice.

FIG. 11 SHARP generating 6000 and 7923 bp amplicons. The 6000 bp product has high yield. Generating 7929 amplicon is possible with SHARP, the product amount is much lower than for 6000 bp amplicon. the 7929 product was amplified only a few times, while 6000 bp product was amplified exponentially.

FIGS. 12A-12C: SHARP amplification of genomic DNA (gDNA) from human cells. FIG. 12A: Monitoring the amount of amplification product in real time. SHARP detects the correct sequence in under 30 minutes. Nonspecific amplification in control experiment (-gDnA) occurred after 45 minutes giving a clear separation in time between the specific and nonspecific product. FIG. 12B: Gel-based assays compares SHARP and PCR product for both genes. FIG. 10C: Both SHARP and PCR product can be sequenced in the same manner to confirm the amplification of the correct gene.

FIG. 13 Amplification of repetitive sequence, 4 repeats of 601 nucleosome positioning sequence with a linker (65 or 80 bp), showed truncated products and smears in PCR but not in SHARP (unpublished data), likely because in SHARP, the DNA is not globally melted which would otherwise cause pairing with internal segments.

### DETAILED DESCRIPTION

A novel method named herein as SHARP (SSB-Helicase Assisted Rapid Polymerase chain reaction) uses helicase and SSB (Single-Stranded Binding protein) to initiate, and DNAP (DNA polymerase) to carry out the isothermal amplification reaction.

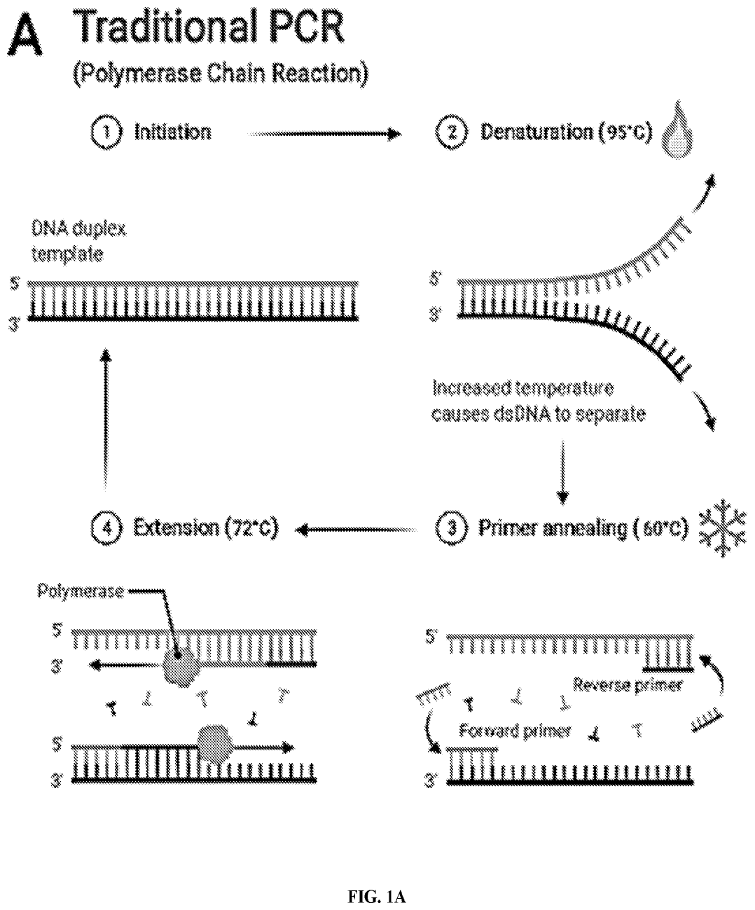

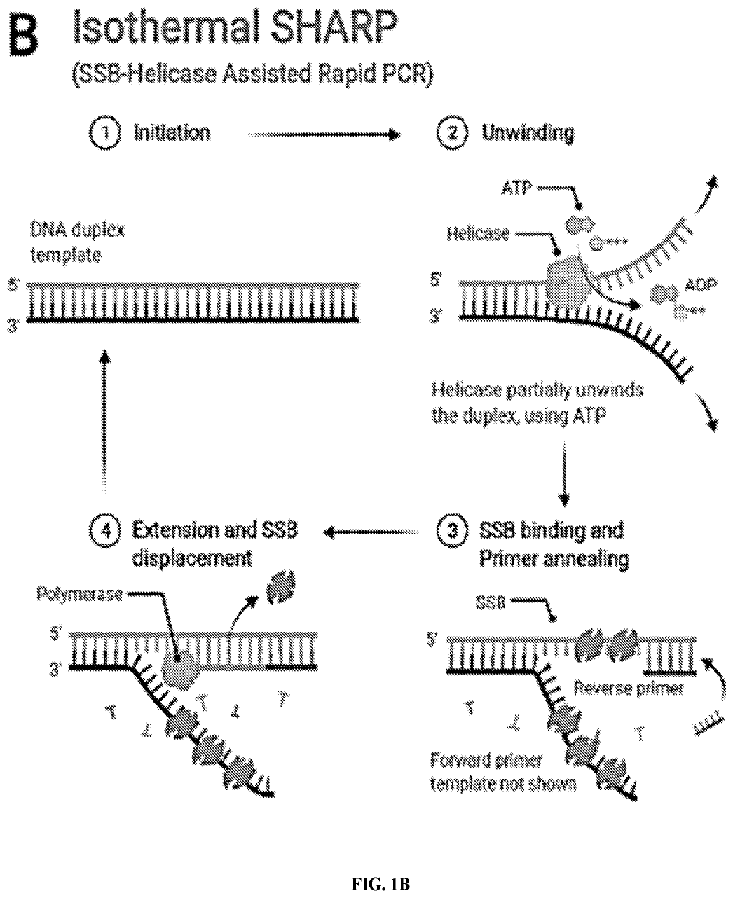

Amplification methods overview: PCR is a fast and sensitive technique to amplify or copy segments of DNA starting from the initially small amount of DNA (3, 4) as the first step in any downstream detection or application. PCR alternates the temperature between high (≈98° C.) to melt the DNA duplex and low (45 to 72° C.) to replicate the missing strand by a DNAP (Taq, Pfu, Q5 etc.) (FIG. 1A). Given a template and a set of 2 template-specific primers, each PCR cycle doubles the number of template copies leading to an exponential increase, thereby a macroscopically detectable product after ˜30 cycles. Currently, there are more than 10 different approaches for isothermal amplification (1, 2); however, no existing isothermal method is as universal and versatile as PCR.

PCR and isothermal methods really on a DNAP to carry out the in vitro replication through primer extension (1, 2), but the methods differ in their approach to initiate each subsequent reaction cycle and allow primers to bind the template. PCR heat denatures the DNA duplex and subsequently cools it down to initiate each replication cycle (FIG. 1A), while isothermal methods either use specially designed primers or other assisting proteins to enable primer binding to the template. LAMP (Loop-Mediated Isothermal Amplification) uses 4 to 6 loop-forming primers to generate 3′ ends that are extendable by Bst DNAP (5). LAMP is a rapid, commercially available (6), and highly specific exponential amplification method resulting in a macroscopically detectable product; however, LAMP product is highly complex, hard to interpret, compare, and use in many applications including cloning, sequencing and gene manipulation. LAMP does not amplify a specific region of interest, unlike PCR. Further, LAMP primer design requires specialized software, and primers optimized for PCR cannot be used as LAMP primers.

Isothermal amplification methods based on gp32, a single stranded DNA binding protein, uses a set of two primers, just like PCR, and can produce over 1 kbp amplicons (2); however, the template is amplified only a few times, far from the exponential growth in PCR, and the product also contains high-molecular weight multimers and other undesired bands. RPA (recombinase polymerase amplification) uses two primers and a recombinase (7, 8). RPA primers are sometimes longer than PCR primers, 30 to 38 bases, which is necessary for recombinase binding. The recombinase-primer complex then searches for homologous template sequence resulting in the strand invasion and primer-template pairing, followed by the polymerase extension. RPA provides an exponential amplification and competitive detection limit, but it can produce only short amplicons, 100 to 200 bp in length. Longer primers used by RPA are more prone to forming secondary structure and nonspecific product, requiring more careful primer design, or using unnatural bases (9). SIBA (strand invasion-based amplification) also uses a recombinase, UvsX, but it reduces the possibility of the undesired product formation by using an invasion oligonucleotide in addition to primers (10, 11) but still generates only short amplicons. SDA (strand displacement amplification) (12) and NEAR (nicking enzyme amplification reaction) (13) are similar methods that use a nicking enzyme to assist the amplification initiation. Both methods provide exponential amplification, but the product is also only several hundred base pairs long. Both methods use a set of 4 primers, with additional sequence requirements for the nicking enzyme. HDA or helicase-dependant amplification (14) utilizes a DNA helicase to generate single-stranded templates for primer hybridization and subsequent primer extension by a DNAP. HDA is also commercially available, and it uses similar primers to PCR. Combining UvrD helicase, MutL accessory protein, T4 gene 32 protein (SSB), and Klenow Exo-DNAP, Vincent et al. achieved an exponential amplification of 100 bp product (14); however, for longer amplicons reaction yield dropped significantly, limiting possible applications of the method. Due to the limitations summarized above, existing isothermal methods are used for specific applications, and cannot be used as broadly as PCR (1).

SHARP (SSB-Helicase Assisted Rapid Polymerase chain reaction): SHARP removes thermal cycling from PCR, keeps all other desired PCR features in place, and adds new features. SHARP performs equally well or better than PCR according to 8 criteria we benchmarked against: (1) up to 6000 bp amplicon length, (2) 5 to 30 minutes amplification time, (3) primer design principles and convenience, (4) detection limit, (5) real-time detection, (6) amplification-result interpretation, (7) downstream product applications, and (8) no initial heat-denaturing step. Numerous benefits of removing a thermal cycler from PCR for faster diagnostics are often discussed in the literature (Y. Zhao, et al., “Isothermal Amplification of Nucleic Acids,” Chemical Reviews, vol. 115, p. 12491-12545, 11 2015; Y. Zhang and N. A. Tanner, “Isothermal Amplification of Long, Discrete DNA Fragments Facilitated by Single-Stranded Binding Protein,” Scientific Reports, vol. 7, 8 2017), and here the suitability of isothermal reaction was tested for other common tasks in the wet lab as described in the examples section which follows. Briefly, it was demonstrated that E. coli cells transformed with 3.2 kbp SHARP-made plasmid can replicate the plasmid and display antibiotic resistance; hence, SHARP is suitable for cloning, sequencing and gene manipulation. It was also shown that DNA sequences with potential to form non-canonical structures, such as sequence containing (CAG)47 repeats, do not inhibit SHARP. Because PCR heats the sample up to 98° C., it narrows down possible enzymes one may use to improve the reaction speed, yield, product fidelity, detection specificity, and ability to simultaneously perform other reactions. SHARP works optimally at 65° C., but can be carried out in the range between 37-65° C., leaving many possibilities for future improvements and combination with other techniques.

Accordingly, in certain embodiments, a method of amplifying nucleic acids comprises: mixing a first composition with one or more primers, a target nucleic acid sequence, and a second composition. In certain embodiments, the mixed compositions are incubated for about 20-60 minutes at a constant temperature in a temperature range of between 37° C. to 90° C. In certain embodiments, the mixed composition is incubated for about 30 minutes at a constant temperature of about 65° C. In certain embodiments, the first composition comprises a nucleic acid dye, deoxyribonucleotide triphosphates (dNTPs), adenosine triphosphate (ATP) or combinations thereof. In certain embodiments, the second composition comprises: an engineered PcrA helicase, a single stranded binding protein (SSB), a Bst polymerase, a thermostable pyrophosphatase (PPase) buffer or combinations thereof. In certain embodiments, the first and second compositions further comprise: water, buffers or the combination thereof.

Accordingly, in certain embodiments, a bacterial derived helicase comprises a PcrA helicase having at least two or more mutations. In certain embodiments, the PcrA helicase comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 90%, 91%, 92%, 93%, 94%, 95%, 96%, 97%, 98%, or 99%, sequence identity to SEQ ID NO: 1. In certain embodiments, the PcrA helicase comprises at least a 95%, 96%, 97%, 98%, or 99%, sequence identity to SEQ ID NO: 1. In certain embodiments, the PcrA helicase comprises SEQ ID NO: 1. In certain embodiments, the PcrA helicase is derived from Geobacillus. In certain embodiments, the Geobacillus is Geobacillus stearothermophilus.

In certain embodiments, a vector encodes a PcrA helicase, wherein the PcrA helicase comprises a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 90%, 91%, 92%, 93%, 94%, 95%, 96%, 97%, 98%, or 99%, sequence identity to SEQ ID NO: 1. In certain embodiments, the PcrA helicase comprises at least a 95%, 96%, 97%, 98%, or 99%, sequence identity to SEQ ID NO: 1. In certain embodiments, the PcrA helicase comprises SEQ ID NO: 1. In certain embodiments, the PcrA helicase is derived from Geobacillus stearothermophilus.

In certain embodiments, a vector comprises a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, a vector comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, a vector comprises at least an 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, a vector comprises at least a 90%, 91%, 92%, 93%, 94%, 95%, 96%, 97%, 98%, or 99%, sequence identity to SEQ ID NO: 2. In certain embodiments, a vector comprises SEQ ID NO: 2.

In certain embodiments, a nucleic acid sequence comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 3. In certain embodiments, the nucleic acid sequence comprises at least a 75%, 80%, 85%, 86%, 87%, 88%, 89%, 90%, 91%, 92%, 93%, 94%, 95%, 96%, 97%, 98%, or 99% sequence identity to SEQ ID NO: 3. In certain embodiments, the nucleic acid sequence comprises at least a 90%, 91%, 92%, 93%, 94%, 95%, 96%, 97%, 98%, or 99% sequence identity to SEQ ID NO: 3. In certain embodiments, a nucleic acid sequence comprises SEQ ID NO: 3. In certain embodiments, SEQ ID NO: 3 encodes an amino acid comprising amino acid substitutions at positions H93A, C96A, N187C, C247A, L384V and L409C.

In certain embodiments, an engineered helicase comprises a PcrA helicase wherein said PcrA helicase comprises one or more amino acid mutations. In certain embodiments, the PcrA helicase is derived from Geobacillus stearothermophilus. In certain embodiments, the PcrA helicase comprises mutations at amino positions 93, 96, 187, 247, 384 and 409 of wild type PcrA helicase.

SHARP enables new generation of portable, point-of-care, point-of-need, and home medical molecular diagnostics tests. Traditional PCR requires a thermocycler, a substantial and high-power-consuming device, and it heats biomolecules above 95° C., making PCR hardly available outside specialized labs. SHARP uses bioengineered enzymes to eliminate the need for complex hardware. SHARP can be carried out with a simple low power heater; hence, molecular diagnostics tests can be performed where needed. SHARP can be used in applications for detecting, for example: infectious diseases, infectious disease agents, disease agents, cancer screening, sepsis, veterinary medicine, agriculture and food processing, forestry cannabis research, and as a general biotechnology tool instead of PCR.

### Helicase Enzymes

A traditional definition of a helicase is an enzyme that catalyzes the reaction of separating/unzipping/unwinding the helical structure of nucleic acid duplexes (DNA, RNA or hybrids) into single-stranded components, using nucleoside triphosphate (NTP) hydrolysis as the energy source (such as ATP). However, it should be noted that not all helicases fit this definition anymore. A more general definition is that they are motor proteins that move along the single-stranded or double stranded nucleic acids (usually in a certain direction, 3′ to 5′ or 5 to 3, or both), i.e., translocases, that can or cannot unwind the duplexed nucleic acid encountered. In addition, some helicases simply bind and “melt” the duplexed nucleic acid structure without an apparent translocase activity.

Helicases exist in all living organisms and function in all aspects of nucleic acid metabolism. Helicases are classified based on the amino acid sequences, directionality, oligomerization state and nucleic-acid type and structure preferences. The most common classification method was developed based on the presence of certain amino acid sequences, called motifs. According to this classification helicases are divided into 6 super families: SF1, SF2, SF3, SF4, SF5 and SF6. SF1 and SF2 helicases do not form a ring structure around the nucleic acid, whereas SF3 to SF6 do. Superfamily classification is not dependent on the classical taxonomy.

DNA helicases are responsible for catalyzing the unwinding of double-stranded DNA (dsDNA) molecules to their respective single-stranded nucleic acid (ssDNA) forms. Although structural and biochemical studies have shown how various helicases can translocate on ssDNA directionally, consuming one ATP per nucleotide, the mechanism of nucleic acid unwinding and how the unwinding activity is regulated remains unclear and controversial (T. M. Lohman, E. J. Tomko, C. G. Wu, “Non-hexameric DNA helicases and translocases: mechanisms and regulation,” Nat Rev Mol Cell Biol 9:391-401 (2008)). Since helicases can potentially unwind all nucleic acids encountered, understanding how their unwinding activities are regulated can lead to harnessing helicase functions for biotechnology applications.

The term “HDA” refers to Helicase Dependent Amplification, which is an in vitro method for amplifying nucleic acids by using a helicase preparation for unwinding a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification.

The SHARP method utilizes a novel PcrA M6 helicase enzyme which was engineered to eliminate a thermal cycling machine from Polymerase Chain Reaction (PCR), while retaining all PCR characteristics.

The amino acid sequence for PcrA M6 comprises the following mutations: H93A,
C96A, N187C, C247A, L384V, L409C (SEQ ID NO: 1):
MNFLSEQLLAHLNKEQQEAVRTTEGPLLIMAGAGSGKTRVLTHRIAYLMAEKHVAP
WNILAITFTNKAAREMRERVQSLLGGAAEDVWISTFASMAVRILRRDIDRIGINRNFSILD
PTDQLSVMKTILKEKNIDPKKFEPRTILGTISAAKNELLPPEQFAKRASTYYEKVVSDVY
QEYQQRLLRCHSLDFDDLIMTTIQLFDRVPDVLHYYQYKFQYIHIDEYQDTNRAQYTLV
KKLAERFQNIAAVGDADQSIYRWRGADIQNILSFERDYPNAKVILLEQNYRSTKRILQAA
NEVIEHNVNRKPKRLWTENPEGKPILYYEAMNEADEAQFVAGRIREAVERGERRYRDF
AVLYRTNAQSRVMEEMLLKANIPYQIVGGVKFYDRKEIKDILAYLRVIANPDDDCSLLRI
INVPKRGIGASTIDKLVRYAADHELSLFEALGELEMIGLGAKAAGALAAFRSQLEQWTQ
LQEYVSVTELVEEVLDKSGYREMLKAERTIEAQSRLENLDEFLSVTKHFENVSDDKSLIA
FLTDLALISDLDELNGTEQAAEGDAVMLMTLHAAKGLEFPVVFLIGMEEGIFPHNRSLE
DDDEMEEERRLAYVGITRAEEELVLTSAQMRTLFGNIQMNPPSRFLNEIPAHLLETASRR
QAGASRPAVSRPQASGAVGSWKVGDRANHRKWGIGTVVSVRGGGDDQELDIAFPSPIG
IKRLLAKFAPIEKV
The plasmid sequence for PcrA M6 H93A, C96A, N187C, C247A, L384V,
L409C (Vector name: pET-11b,Cloning site: Ndel/BamHI, Variant sequence includes N terminal
6xHis tag and C-terminal FLAG tag) SEQ ID NO: 2:
catatgcatcaccatcaccatcacgggtccggaatgaact ttttatcggaacagctgctcgcccatttaaacaaagagcaacaag
aagccgtcaggacgacggaaggcccgctgctcattatggcggggg cgggaagcgggaaaacgcgggtgttga
cgcaccgcatcgcctatt tgatggcggaaaagcatgtggcgccgtggaacattttggccatta cgtttacgaacaag
gcggcgcgcgaaatgcgggaacgtgtgcagt cgctcttaggtggggcggcggaagacgtctggatttcgacgttcg
cgtcgatggccgtccgcattttgcgccgcgacattgaccgcatcg gcatcaaccgcaatttttccatccttgatccgacggaccagcttt
cagtcatgaaaacgattttaaaagaaaaaaacatagacccgaaaa aatttgagccgcggacgattttaggcacgatcagcgcggcgaaaa
acgagctgttgcctccggagcaattcgcgaagcgggcctcgacgt attacgaaaaagtcgtcagcgatgtgtatcaagaataccaacagc
gcctgcttcgctgtcattcgctcgattttgacgatttgatcatga cgacgatccaactgtttgaccgcgtgccggatgtgcttcactatt
accaatataagtttcagtacattcatattgatgagtaccaggata cgaaccgcgctcaatatacgctggtcaaaaagctggcggaacgct
ttcaaaacattgccgccgtcggcgacgccgaccaatcgatttatc ggtggcgcggggcggacatccaaaacattttgtcgttcgagcgcg
actatccgaacgcaaaagtcattttgcttgaacaaaactaccgct cgacgaagcgcattttgcaagcggcgaacgaagtcatcgagcata
acgtcaaccggaagccgaaacggctttggacggaaaacccggaag gaaagccgattctttattatgaggcgatgaac
gaagcggacgaag cgcagtttgtcgctggacgcatccgcgaggcggtggagcgcggcg aacgccgctacc
gtgattttgctgtcttgtaccggacgaacgccc agtcgcgtgtcatggaggaaatgttgctgaaagcgaacattccgt
atcaaattgtcggcggcgtaaagttctatgaccggaaagaaatta aagacattctcgcctatttgcgcgtcattgccaatccggacgatg
attgcagcttgcttcgcatcattaacgtgccaaaacgcggcattg gcgcctcgacgatcgacaaactcgtccgctatgcagccgatcatg
agctgtccttgtttgaggcgctcggcgagctagagatgatcgggc ttggcgccaaagcggccgggg
cgctcgccgcgttccgcagccagc tcgagcaatggacacagctgcaagaatacgtctccgtcaccgaac
tcgtcgaagaagtgctcgacaaatcgggctaccgcgagatgctca aggcggagcggacgattgaagcacaaa gccggctcga
gaacttgg atgagtttttgtcggtgacgaagcattttgaaaatgtgagcgacg ataaatcgctcatcgcctttttaaccgac
ttggcgctcatttccg atttggacgagctgaacgggacggaacaggccgctgaaggagatg ccgtcatgttgatgacgttgcatgccgc
caaagggctcgagtttc cggtcgtctttttgatcggcatggaagaaggcattttcccgcaca accgctctctcgaggatgacgatga
gatggaagaagaacggggc tggcgtacgtcggcatcacccgcgcggaggaagaacttgtgctga cgagcgcgcaaatgcggac
gttgtttggcaacatccaaatgaacccgccgtcgcgctttttgaatgaaattccggcgcatttgcttgaga cagcctcgcgccgccaa
gcgggcgcctcccgcccggccgtttcgc gcccgcaggcaagcggcgccgtgggatcgtggaaagtcggcgatc
gggcgaatcaccggaaatggggcatcggcaccgtcgtcagcgtcc gcggcggcggcgacgaccaa gagctcgaca
tcgccttcccgagcc cgatcggcattaaacggttgcttgccaaatttgcgccgattgaga aagtgggtagcggcgacta
caaggacgatgacgataaggattata aagacgatgacgataaggactacaaagacgatgacgataaatagg gatcc
The amino acid sequence for the wild type PcrA (NCBI Reference Sequence
WP_033016687.1) is listed below and available on: ncbi.nlm.nih.gov/protein/WP_033016687.1
(SEQ ID NO: 3):
MNFLSEQLLAHLNKEQQEAVRTTEGPLLIMAGAGSGKTRVLTHRIAYLMAEKHVAP
WNILAITFTNKAAREMRERVQSLLGGAAEDVWISTFHSMCVRILRRDIDRIGINRNFSILD
PTDQLSVMKTILKEKNIDPKKFEPRTILGTISAAKNELLPPEQFAKRASTYYEKVVSDVY
QEYQQRLLRNHSLDFDDLIMTTIQLFDRVPDVLHYYQYKFQYIHIDEYQDTNRAQYTLV
KKLAERFQNICAVGDADQSIYRWRGADIQNILSFERDYPNAKVILLEQNYRSTKRILQAA
NEVIEHNVNRKPKRLWTENPEGKPILYYEAMNEADEAQFVAGRIREAVERGERRYRDF
AVLYRTNAQSRVMEEMLLKANIPYQIVGGLKFYDRKEIKDILAYLRVIANPDDDLSLLRI
INVPKRGIGASTIDKLVRYAADHELSLFEALGELEMIGLGAKAAGALAAFRSQLEQWTQ
LQEYVSVTELVEEVLDKSGYREMLKAERTIEAQSRLENLDEFLSVTKHFENVSDDKSLIA
FLTDLALISDLDELNGTEQAAEGDAVMLMTLHAAKGLEFPVVFLIGMEEGIFPHNRSLE
DDDEMEEERRLAYVGITRAEEELVLTSAQMRTLFGNIQMNPPSRFLNEIPAHLLETASRR
QAGASRPAVSRPQASGAVGSWKVGDRANHRKWGIGTVVSVRGGGDDQELDIAFPSPIG
IKRLLAKFAPIEKV

Accordingly, in certain embodiments, a bacterial derived helicase comprising a PcrA helicase having at least two or more mutations. In certain embodiments, the PcrA helicase comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the PcrA helicase comprises at least a 90%, 91%, 92%, 93%, 94%, 95%, 96%, 97%, 98%, or 99%, sequence identity to SEQ ID NO: 1. In certain embodiments, the PcrA helicase comprises at least a 95%, 96%, 97%, 98%, or 99%, sequence identity to SEQ ID NO: 1. In certain embodiments, the PcrA helicase comprises SEQ ID NO: 1.

In certain embodiments, the PcrA helicase is derived from Geobacillus. In certain embodiments, the Geobacillus is Geobacillus stearothermophilus. In certain embodiments, a nucleic acid sequence, SEQ ID NO: 3, encodes an amino acid comprising amino acid substitutions at positions H93A, C96A, N187C, C247A, L384V and L409C.

In certain embodiments, an engineered helicase comprising a PcrA helicase wherein said PcrA helicase comprises one or more amino acid mutations. In certain embodiments, the PcrA helicase is derived from Geobacillus stearothermophilus. In certain embodiments, the PcrA helicase comprises mutations at amino positions 93, 96, 187, 247, 384 and 409 of wild type PcrA helicase.

In certain embodiments, the mutations at positions 93, 96, 187, 247, 384 and 409 of wild type PcrA helicase comprise both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

In certain embodiments, the PcrA helicase comprises one or more modified peptide bonds. As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S——CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. I 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)).

Vectors: In certain embodiments, a vector encodes for a PcrA helicase comprising one or more mutations. In certain embodiments, a vector encodes a PcrA helicase comprising a 60% sequence identity to SEQ ID NO: 1. In certain embodiments, the PcrA helicase comprises at least a 75% sequence identity to SEQ ID NO: 1. In certain embodiments, the PcrA helicase comprises at least a 90% sequence identity to SEQ ID NO: 1. In certain embodiments, the PcrA helicase comprises at least a 95% sequence identity to SEQ ID NO: 1. In certain embodiments, the PcrA helicase comprises SEQ ID NO: 1. In certain embodiments, the PcrA helicase is derived from Geobacillus stearothermophilus.

In certain embodiments a vector comprises the sequence, SEQ ID NO: 2.

The nucleic acids encoding the PcrA polypeptides can be adapted to suitable expression systems for producing the helicase polypeptides for PcrA helicase production. For DNAs encoding PcrA helicase genes, the representative genes can be operably-linked to suitable expression vectors for expressing the proteins in bacterial, fungal, insect or other suitable expression host. For RNAs encoding PcrA helicase polypeptides, the representative RNAs can be engineered for enabling efficient expression in vitro of the polypeptides in extract lysates produced from bacterial, fungal, insect or other suitable expression host sources. Such systems are well known in the art. Following expression, the PcrA helicase polypeptides can be purified by methods known in the art, including affinity-tag chromatography, SDS-PAGE, and size-exclusion chromatography, among others.

In certain embodiments, vectors such as, for example, expression vectors, containing a nucleic acid encoding one or more PcrA helicase polypeptides described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably. However, this disclosure is intended to include such other forms of expression vectors, such as viral vectors (e.g., replication defective retroviruses, adenoviruses and adeno-associated viruses), which serve equivalent functions.

In certain embodiments, the recombinant expression vectors comprise a nucleic acid sequence (e.g., a nucleic acid sequence encoding one or more PcrA helicase polypeptides described herein) in a form suitable for expression of the nucleic acid sequence in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence encoding one or more PcrA helicase polypeptides is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors described herein can be introduced into host cells to thereby produce proteins or portions thereof, including fusion proteins or portions thereof, encoded by nucleic acids as described herein (e.g., one or more PcrA helicase polypeptides).

Recombinant expression vectors can be designed for expression of one or more encoding one or more PcrA helicase polypeptides in prokaryotic or eukaryotic cells. For example, one or more vectors encoding one or more PcrA helicase polypeptides can be expressed in bacterial cells such as E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40); pMAL (New England Biolabs, Beverly, Mass.); and pRIT5 (Pharmacia, Piscataway, N.J.) which fuse glutathione S-transferase (GST), maltose E binding protein, or protein A, respectively, to the target recombinant protein.

In another embodiment, the expression vector encoding one or more PcrA helicase polypeptides is a yeast expression vector. Examples of vectors for expression in yeast S. cerevisiae include pYepSec1 (Baldari, et. al., (1987) EMBO J. 6:229-234); pMFa (Kurjan and Herskowitz, (1982) Cell 30:933-943); pJRY88 (Schultz et al., (1987) Gene 54:113-123); pYES2 (Invitrogen Corporation, San Diego, Calif.); and picZ (Invitrogen Corporation).

Alternatively, one or more PcrA helicase polypeptides can be expressed in insect cells using baculovirus expression vectors. Baculovirus vectors available for expression of proteins in cultured insect cells (e.g., Sf9 cells) include the pAc series (Smith et al. (1983) Mol. Cell. Biol. 3:2156-2165) and the pVL series (Lucklow and Summers (1989) Virology 170:31-39).

In certain embodiments, a nucleic acid described herein is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, adenovirus 2, cytomegalovirus and simian virus 40. For other suitable expression systems for both prokaryotic and eukaryotic cells see Green M., and Sambrook; J. Molecular Cloning: A Laboratory Manual. 4th, ed., Cold Spring Harbor Laboratory, Cold Spring Harbor Laboratory Press, Cold Spring Harbor, N.Y., 2012.

In certain embodiments, host cells into which a recombinant expression vector of the invention has been introduced are provided. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, one or more PcrA helicase polypeptides can be expressed in bacterial cells such as E. coli, viral cells such as retroviral cells, insect cells, yeast or mammalian cells (such as Chinese hamster ovary cells (CHO) or COS cells). Other suitable host cells are known to those skilled in the art.

Delivery of nucleic acids described herein (e.g., vector DNA) can be by any suitable method in the art. For example, delivery may be by injection, gene gun, by application of the nucleic acid in a gel, oil, or cream, by electroporation, using lipid-based transfection reagents, or by any other suitable transfection method.

As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAF-dextran-mediated transfection, lipofection (e.g., using commercially available reagents such as, for example, LIPOFECTIN™ (Invitrogen Corp., San Diego, Calif.), LIPOFECTAMINE™ (Invitrogen), FUGENE™ (Roche Applied Science, Basel, Switzerland), JETPEI™ (Polyplus-transfection Inc., New York, N.Y.), EFFECTENE™ (Qiagen, Valencia, Calif.), DREAMFECT™ (OZ Biosciences, France) and the like), or electroporation (e.g., in vivo electroporation). Suitable methods for transforming or transfecting host cells can be found in Green and Sambrook, et al. (Molecular Cloning: A Laboratory Manual. 4th, ed., Cold Spring Harbor Laboratory, Cold Spring Harbor Laboratory Press, Cold Spring Harbor, N.Y., 2012), and other laboratory manuals.

### Kits

In certain embodiments, a kit comprises an engineered PcrA helicase, a single stranded binding protein (SSB), a polymerase, a thermostable pyrophosphatase (PPase). In certain embodiments, the engineered PcrA helicase comprises SEQ ID NO: 1. In certain embodiments, the polymerase comprises: DNA polymerase (DNAP) I, DNAP II, DNAP III, DNAP IV, DNAP V, Klenow fragment, reverse transcriptases, Exo-polymerase, high-fidelity polymerases, Taq polymerases, bacteriophage polymerases, Pyrococcus furiosus Vc1 (Pfu) polymerase, Hot Start DNA Polymerase, or eukaryotic polymerases. In certain embodiments, the polymerase is a Geobacillus stearothermophilus DNA polymerase I, Bacillus subtilis DNA polymerase I, Klenow fragment or Exo-polymerase. In certain embodiments, the Geobacillus stearothermophilus DNA polymerase I is Bst polymerase or Bst-large fragment polymerase (Bst-LF). In certain embodiments, the reverse transcriptase is murine leukemia virus (MMLV) reverse transcriptase.

In certain embodiments, the kit further comprises buffers, deoxyribonucleotide triphosphates (dNTPs), nucleoside triphosphates (NTPs), a detectable nucleic acid label, water, dithiothreitol (DTT) or combinations thereof. In certain embodiments, the detectable nucleic acid label comprises a fluorescent dye.

In certain embodiments, a kit comprises a first composition and a second composition. In certain embodiments, the first composition comprises: a nucleic acid dye, deoxyribonucleotide triphosphates (dNTPs), adenosine triphosphate (ATP) or combinations thereof. In certain embodiments, the second composition comprises: an engineered PcrA helicase, a single stranded binding protein (SSB), a polymerase, a thermostable pyrophosphatase (PPase) buffer or combinations thereof. In certain embodiments, the polymerase comprises: DNA polymerase (DNAP) I, DNAP II, DNAP III, DNAP IV, DNAP V, Klenow fragment, reverse transcriptases, Exo-polymerase, high-fidelity polymerases, Taq polymerases, bacteriophage polymerases, Pyrococcus furiosus Vc1 (Pfu) polymerase, Hot Start DNA Polymerase, or eukaryotic polymerases. In certain embodiments, the polymerase is a Geobacillus stearothermophilus DNA polymerase I, Bacillus subtilis DNA polymerase I, Klenow fragment or Exo-polymerase. In certain embodiments, the Geobacillus stearothermophilus DNA polymerase I is Bst polymerase or Bst-large fragment polymerase (Bst-LF). In certain embodiments, the reverse transcriptase is murine leukemia virus (MMLV) reverse transcriptase.

In certain embodiments, the kit further comprises buffers, deoxyribonucleotide triphosphates (dNTPs), nucleoside triphosphates (NTPs), a detectable nucleic acid label, water, dithiothreitol (DTT) or combinations thereof. In certain embodiments, the detectable nucleic acid label comprises a fluorescent dye.

In certain embodiments, the first and second compositions further comprise; water, buffers or the combination thereof. In certain embodiments, first composition is contained in a first vial and the second composition is contained within a second vial.

In certain embodiments, the kit may further comprise a helicase, a primer pair, a polymerase, and optionally, a detection system for detecting amplification of the target nucleic acid, as described herein. In certain embodiments, the primer pair may comprise a first and second primer, with the first primer comprising a portion that is complementary to the first strand of the target nucleic acid and the second primer comprising a portion that is complementary to the second strand of the target nucleic acid. The kit may also include a set of instructions for use. In certain embodiments, the kit may include reagents for purifying the double-stranded nucleic acid in the sample. In some embodiments, the kit may be a kit for amplifying and/or detecting a target single-stranded nucleic acid in a sample and may include reagents for purifying the single-stranded nucleic acid in the sample. The kit may also include a set of instructions for use.

### EXAMPLES

### Example 1: Rapid Isothermal Amplification of Several Kilo Base Pair DNA

A new robust isothermal amplification method is described herein, which is analogous to PCR in that it uses the same set of primers and template as PCR at the input. Further, the method outputs the same amplicon as PCR, with the length of up to 6000 base pairs (bp) but is performed at a constant temperature, thus obviating the need for thermal cycling. This new method is named SHARP (SSB-Helicase Assisted Rapid Polymerase chain reaction) herein, because it uses helicase and SSB (Single-Stranded Binding protein) to initiate, and DNAP (DNA polymerase) to carry out the isothermal amplification reaction.

### Materials and Methods

SHARP reaction mix: Three principal enzymes, Bst-LF DNAP, E.coli SSB, and PcrA M6 helicase, were overexpressed and purified. The following enzymes were used for SHARP with stock concentrations: SSB (9 mg/mL), PcrA helicase (0.2 mg/mL), Bst-LF DNAP (1.5 mg/mL), PPase (2000 units/mL, NEB catalog #M0296S). Other stock components are dNTP's (10 mM each), ATP (100 mM), Evagreen dye (20× from Biotium #31000), DTT (Dithiothreitol, 100 mM in water). The 10× reaction buffer contains 500 mM Potassium acetate, 200 mM Tris-acetate, 100 mM Magnesium acetate, 1 mg/mL BSA, pH 7.9. Primer stock concentrations are at 10 or 20 μM, while the template concentration is variable.

For each SHARP reaction, 40 μL total volume was prepared and the volume was split to 2 separate wells on a 96-well plate to have 2 independent fluorescence readouts of the same reaction. SYBR (EvaGreen) intensity was monitored in real time every 10 s for each well on BioRad CFX96 machine. Table 1 contains raw component volumes.

TABLE 1
SHARP components for 40 μL reaction volume.

| Component 1 (2X): FuelMix | μL | Component 2 (2X): EnzMix | μL |
|---|---|---|---|
| Water | 9 | Water | 11.1 |
| 10X Buffer | 2 | 10X Buffer | 2 |
| EvaGreen (20X) | 2 | DTT (100 mM) | 4 |
| dNTPs (10 mM each) | 2 | Bst-LF (1.5 mg/mL) | 0.2 |
| ATP (100 mM) | 2 | SSB (9 mg/ml) | 0.5 |
| Forward primer (10 uM) | 1 | PcrA (0.2 mg/mL) | 2 |
| Reverse primer (10 uM) | 1 | Thermostable PPase (100 | 0.2 |
| | | [symbol] | |
| Template (variable conc.) | 1 | Total: | 20 |
| Total: | 20 | | |

[symbol]

indicates data missing or illegible when filed

Component 1 and Component 2 were separately prepared and were mixed together on the plate, before the temperature was increased to 65 degrees and the recording of fluorescence starts. After incubation, the product with was purified with Qiagen PCR cleanup kit and test the product on the gel.

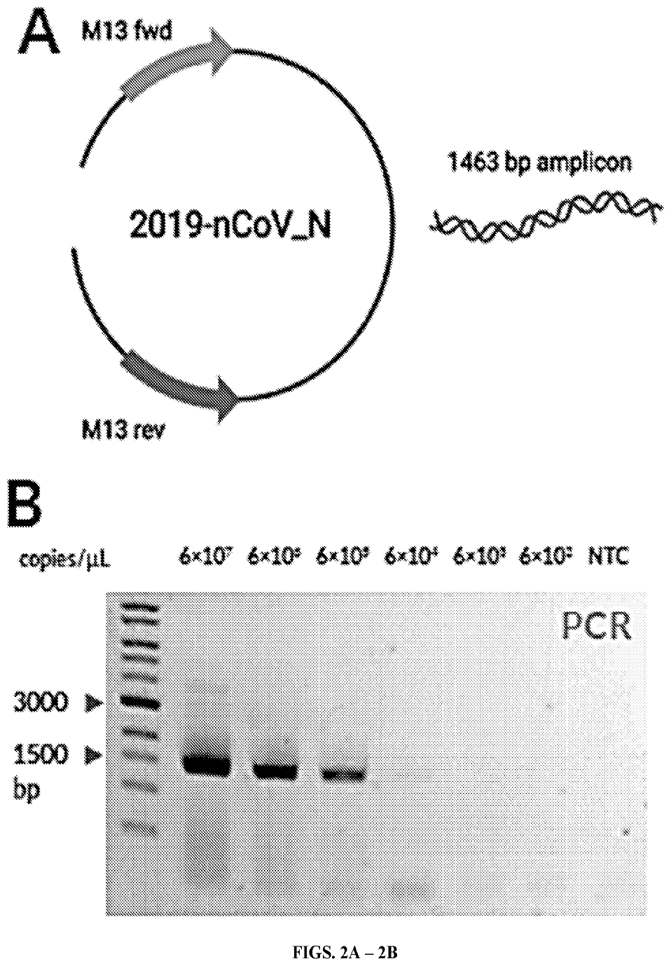

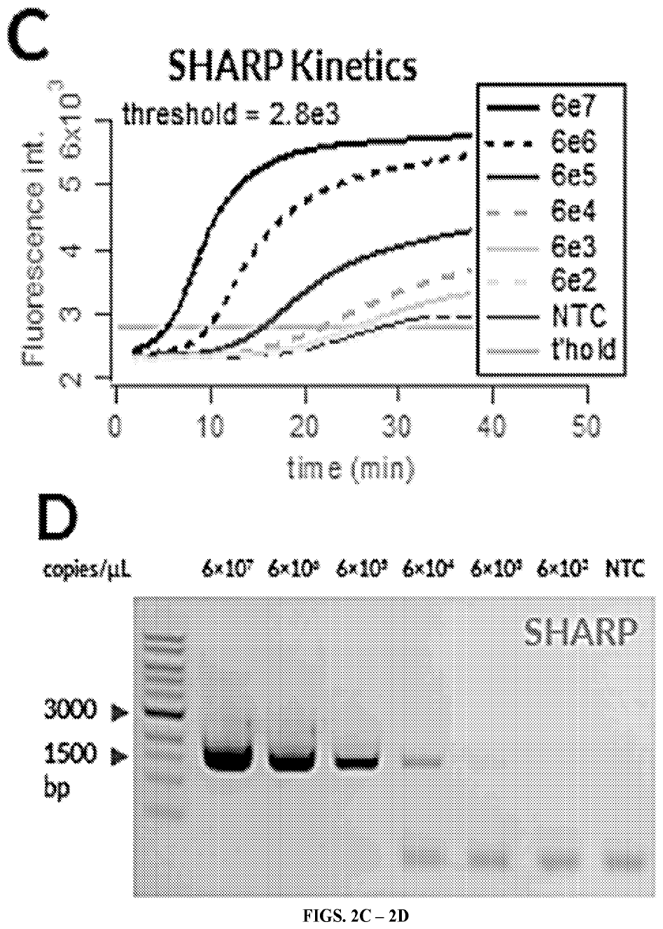

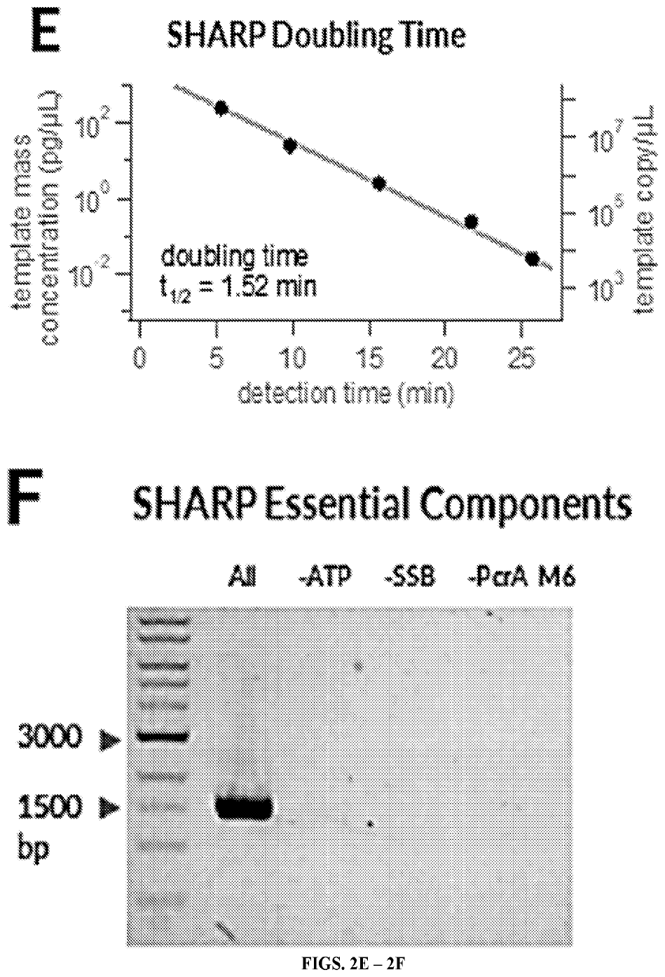

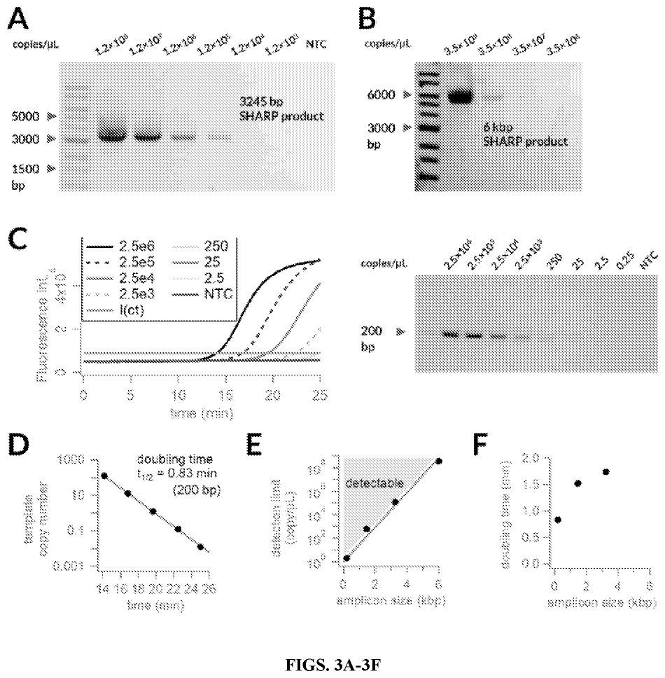

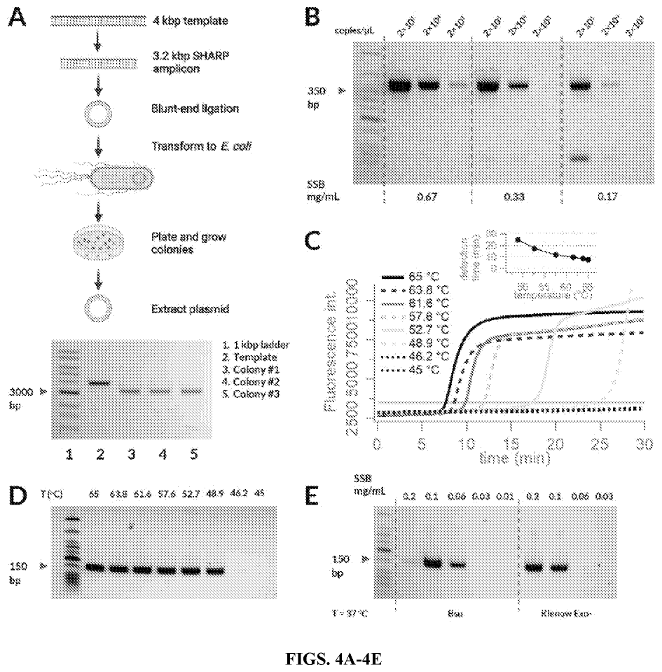

Primers and template: Primers were ordered from Integrated DNA technologies (IDT). DNA template vector containing 2019 coronavirus nCOV-2 N protein sequence (2019-nCOV_N_Positive Control, Catalog #10006625) was also ordered from IDT. For PCR and SHARP in FIGS. 2A-2D and 3C, M13 primers were used:
5′-CCCAGTCACGACGTTGTAAAACG (forward; SEQ ID NO: 4) and
5′-AGCGGATAACAATTTCACACAGG (reverse; SEQ ID NO: 5) and nCOV-2 N template. In FIGS. 2E and 3A the following primers were used:
5′-AATTTTGGGGACCAGGAAC (forward; SEQ ID NO: 6) and
5′-TCTGGTTACTGCCAGTTGAATCTG (reverse; SEQ ID NO: 7) and nCOV-2 N template, as such primers include the vector backbone containing ampicillin resistance. The t-DNA amplicon in FIG. 2G uses t-DNA from NEB (catalog #N3011S) as a template and following primers generating 200 bp amplicon:
5′-CGGCTTCTGACTCTCTTTCC (forward; SEQ ID NO: 7) and
5′-TTCCTTCAAGCTTTGCCACA (reverse; SEQ ID NO: 8). For testing SHARP with CAG repetitive sequence in FIG. 4B, pBluescript-CTG-47 sequence was used from A. Jain and R. D. Vale (“RNA phase transitions in repeat expansion disorders,” Nature, vol. 546, p. 243-247, 5 2017) deposited on Addgene #99150 and M13 primers given above. For testing the temperature dependence of SHARP in FIG. 4C, nCOV-2 N template and the following primers were used:
5′-AATTTTGGGGACCAGGAAC (forward; SEQ ID NO: 9) and
5′-GCACCTGTGTAGGTCAAC (reverse; SEQ ID NO: 10) producing a 155 bp amplicon.

DNA unwinding assays: The FRET unwinding assay uses the following buffer: 10 mM TRIS pH8.0, 10 mM MgCl2, 50 mM NaCl, 1 mM ATP, 1% BSA. DNA concentration is at 5 nM and the helicase concentration varies. The reaction is performed in 0.2 mL cuvette. The spectrofluorometer excites the sample at 550 nm and records the fluorescence at 570 nm (green, Cy3) and 667 nm (red, Cy5). Unwinding of longer DNA substrates in the presence of EvaGreen intercalating dye is carried out in 20 μL volume on qPCR machine in the same buffer as the SHARP reaction (50 mM Potassium acetate, 20 mM Tris-acetate, 10 mM Magnesium acetate, 0.1 mg/mL BSA, pH 7.9). The reaction mix is prepared on ice, and the qPCR machine is precooled to 4° C. Upon placing the same on qPCR machine, the machine starts recording and then rapidly increases the temperature to 37° C.

Protein overexpression and purification: PcrA M6 helicase with 6xHis tag: PcrA M6 helicase, PcrA and all mutants are purified as described previously (Arslan, R. et al. “Engineering of a superhelicase through conformational control,” Science, vol. 348, p. 344-347, 4 2015; J. Park, et al., “PcrA Helicase Dismantles RecA Filaments by Reeling in DNA in Uniform Steps,” Cell, vol. 142, p. 544-555, 8 2010) using a standard Ni-NTA purification column, followed by single-stranded DNA cellulose column. Briefly, the pET-11b vector containing PcrA M6 sequence between NdeI and BamHI sites was used. The vector including 6x-His tag on N terminus has been synthetized by GenScript and GenScript synthetized all point mutations. The vector was transformed to E. coli. BL21 (DE3) pLysS. Cells were grown at 37° C. in the presence of ampicillin and chloramphenicol, moved to 18° C. when OD₆₀₀ reached 0.3 and induced at OD₆₀₀=0.5 with 0.5 mM IPTG and harvested after an overnight incubation at 18° C. Cell pellets, previously stored at −80° C. were resuspended in the lysis buffer (50 mM Tris, 5 mM Imidazole, 200 mM NaCl, 20% Sucrose, 15% Glycerol, 0.5 mg/mL Lysozyme, pH 7.6) and sonicated followed by centrifugation at 35,000 g. The Ni-NTA agarose resin is preequilibrated with the wash buffer (50 mM Tris, 5 mM Imidazole, 150 mM NaCl, 25% (v/v) Glycerol, pH 7.6). 40 mL of cell lysate supernatant is added to 2 mL of equilibrated Ni-NTA resin and incubated for 1 hour at 4° C. with constant stir mixing by inverting the 50 ml tube. After 1 hour, the resin is gently centrifuged at 1000 g for 2 minutes and supernatant is discarded carefully, and the tube with the resin refilled with the wash buffer. The batch wash is repeated 3 times, protein-loaded resin is then poured into a disposable gravity flow column, washed with 20 mL of wash buffer, and eluted with elution buffer made by dissolving 200 mM imidazole in wash buffer. The protein is then loaded to single-stranded DNA cellulose column, washed with buffer (100 mM NaCl, 50 mM Tris, 1 mM EDTA, 20% (v/v) Glycerol), and eluted with (1 M NaCl, 50 mM Tris, 1 mM EDTA, 20% (v/v) Glycerol). The presence of the 6xHis does not affect any downstream application. The protein concentration was always kept below 4 mg/ml (≈50 mM) to avoid aggregation, and the final PcrA protein was stored at −80° C. or −20° C. in the storage buffer containing 600 mM NaC1, 50 mM TRIS pH 7.6, and 50% glycerol. This protocol leads to a diluted PcrA to between 0.2 and 1 mg/mL, if necessary one can concentrate protein with a membrane filtering.

PcrA helicase without 6xHis tag: Plasmid expressing wild-type PcrA helicase without any tag is a gift from Tim Lohman lab. Obtaining the cell lysate is done in the same way as in the previous protocol. The cell lysate is also spun down, but supernatant is mixed with 0.7 volume of saturated ammonium sulfate solution. Ammonium sulfate precipitates PcrA, and PcrA is collected by spinning at 5000 g. PcrA was suspended in the wash buffer (50 mM Tris, 5 mM Imidazole, 150 mM NaCl, 25% (v/v) Glycerol, pH 7.6), spun down, and the supernatant loaded to ssDNA-cellulose column. The rest of the protocol is the same as for PcrA with the 6xHis tag.

Single Stranded binding protein (SSB): E. coli SSB is purified without tag through polymin-P and ammonium sulfate precipitation, followed by the heparin Sepharose column, similar to the protocol by Lohman et al. (“Large-scale overproduction and rapid purification of the Escherichia coli ssb gene product. Expression of the ssb gene under λ PL control,” Biochemistry, vol. 25, p. 21-25, 1 1986). SSB sequence is cloned into pET21a vector using the NdeI and BamHI sites. The vector was used to transform BL21 (DE3) cells, the colonies were picked, grown at 37° C., overexpression was induced with 0.5 mM IPTG at OD₆₀₀=0.5 and grown for an additional 5 hours and the pellet collected. Overexpression levels were usually very high, often SSB made over 60% of the total cell protein. The cell pellet was resuspended in the lysis buffer (50 mM Tris, 200 mM NaCl, 20% Sucrose, 15% Glycerol, 0.5 mg/mL Lysozyme, pH 7.6) and sonicated, followed by the centrifugation at 35,000 g for 30 minutes. The supernatant containing soluble SSB is collected, and Polymin-P of the final concentration 0.2% was added to the supernatant to precipitate SSB. Precipitated SSB was collected by spinning at 4000 g and then resuspended in buffer containing 50 mM TRIS pH 8.3, 20% glycerol, 1 mM EDTA, 400 mM NaCl, and stir mixed at 4° C. for 30 minutes. To remove undissolved protein, the mixture was centrifuged at 10,000 g for 20 minutes and the supernatant containing soluble SSB was collected. Finally, solid ammonium sulfate was added to give the final concentration of 150 g/L, precipitate SSB, and collect the pellet containing SSB after centrifugation at 12,000 for 30 minutes. The pellet was resuspended in 50 mM TRIS pH 8.3, 20% glycerol, 1 mM EDTA, 200 mM NaCl, stir mixed at 4° C. for 30 minutes, centrifuged at 18,000 g for 20 minutes, and filtered through 200 μm filter. We equilibrate heparin-Sepharose column with a wash buffer (50 mM TRIS pH 8.3, 20% glycerol, 1 mM EDTA), dilute the SSB solution in the wash buffer 5 times and load on the column. Next, the bound SSB was washed with 50 to 100 mL wash buffer and eluted with NaCl gradient from 100 mM to 1 M. The final SSB solution was dialyzed against the storage buffer (20 mM Tris, pH 8.1, 50% Glycerol, 0.5M NaCl, 1 mM EDTA, 1 mM BME). SSB concentrated between 6 and 9 mg/mL was obtained. The SHARP reaction uses highly concentrated SSB; hence, it is important to use SSB stock concentrated above 5 mg/mL.

Bst-LF DNA polymerase: Bst-LF purification protocol uses Ni-NTA resin. Bst-LF DNAP expressing vector available through addgene.org/145799/was used and a modified version of the protocol described in (S. Bhadra, et al. “High-surety isothermal amplification and detection of SARS-COV-2, including with crude enzymes,” 4 2020) was followed for the overexpression and purification. Bst-LF expressing plasmid containing 6xHis tag on N terminus was transformed to expressing cells, select individual colonies, grow medium until OD₆₀₀ reaches 0.5 and overexpression was induced with 200 ng/ml anhydrotetracycline (aTC) or 100 ng/ml of tetracycline. The total culture volume of 500 mL was grown at 25 degrees for 12 hour and spun down to form the pellet kept at −80 degrees. The pellet is resuspended in the Lysis buffer (20 mM Tris pH 7.4, 300 mM NaCl, 0.1% Tween-20, 10 mM imidazole, 1×EDTA-free protease inhibitor tablet, 0.5 mg/mL Lysozyme) in the cold room and sonicated to lyse the cells. Lysed cells were centrifuged at 35,000 g for 30 min at 4° C. and the supernatant was collected. The lysate was heat treated at 65° C. for 10 min, cooled down on ice for 10 min at 4° C., and filtered using 60 mL syringe with 200 μm filter. The Ni-NTA agarose resin was pre-equilibrated with the wash buffer (20 mM Tris, pH 7.4, 300 mM NaCl, 0.1% Tween-20, 40 mM imidazole). 40 mL of filtered lysate is added to 2 mL of equilibrated Ni-NTA resin and incubated for 1 hour at 4° C. with constant stir mixing by inverting the 50 ml tube. After 1 hour, the resin was gently centrifuged at 1000 g for 2 minutes, supernatant was discarded carefully, and the tube with the resin refilled with the wash buffer. The batch wash was repeated 3 times, protein-loaded resin is then poured into a disposable gravity flow column, washed with 10 mL of wash buffer and eluted with a buffer containing 20 mM Tris pH 7.4, 300 mM NaCl, 0.1% Tween-20, 250 mM imidazole in 500 μL fractions. Collected fractions were tested on the gel, and dialyzed against the storage buffer (50% Glycerol, 10 mM Tris pH 7.4, 100 mM KCl, 1 mM DTT, 0.1 mM EDTA, 0.5% Tween-20, 0.5% Triton-X100) overnight. Small aliquots of Bst-LF DNAP concentrated at 1.5 mg/mL are kept at −80° C. for long term storage or at −20° C. for daily use. Optimal Bst-LF concentration in the SHARP reaction is between 0.0015 and 0.0075 mg/mL.

### Results

Reference PCR assay: To introduce SHARP a linearized 4012-bp DNA template vector containing 2019 coronavirus nCOV-2 N protein sequence between M13 primers (FIG. 2A was used. FIG. 1A summarizes the traditional thermal cycler-based PCR reaction serving as a control and reference for introducing the new SHARP method. nCOV-2 N template and two M13 primers at 250 nM were mixed with the commercial NEB (New England Biolabs, #M0531S) Phusion master mix containing PCR components. Thermal cycler alternates the temperature between 98° C. to melt the duplex, 65° C. for primers to bind, and 72° C. to extend primers bound to the template. After 30 cycles, each template molecule is theoretically copied 2³⁰≈10⁹ times, leading to a macroscopically detectable 1463 bp product. From the amounts of single-band PCR amplicon in 2% agarose gel vs. the initial template copy number in the range from 6×10² to 6×10⁷ per microliter (FIG. 2B), it was determined that the reference PCR reaction needs at least 6×10³ copies of the template for gel-based detection of 1463 bp product.

SHARP amplification reaction: SHARP was used to carry out the amplification reaction at 65° C., with the same template copy numbers and the same primer set as the reference PCR. SHARP produces the same 1463 bp amplicon as PCR (FIG. 2D). DNA amplification was monitored in real time using intercalating dyes (FIG. 2C). Principal components of SHARP are 1 template, 2 primers at 250 nM each, dNTPs, Bst-LF DNAP, E. coli SSB, engineered PcrA M6 helicase (see the section below titled Engineering PcrA helicase for SHARP), ATP, and thermostable inorganic pyrophosphatase, see Table 1. Excluding SSB, PcrA M6 or ATP prevents the amplification (FIG. 2F). In the development phase, other polymerases were tested (the large fragment of Bsu, Klenow Exo-, phi29, and reverse transcriptase M-MLV and AMV). They all showed some degree of amplification, but none of these other polymerases satisfied all 8 criteria. FIG. 2C shows SHARP kinetics as a function of the initial template copy number. For 6×10⁷ copies per microliter, the SHARP product was detectable after 5 minutes, while for 6×103 it takes about 22 minutes. The 1463 SHARP product imaged on 2% agarose gel in FIG. 2D reproduces the same bands observed for the reference PCR product (FIG. 2B). The detection limit of SHARP was above 6×10² copies per microliter and more sensitive than the detection limit of 6×10³ for PCR in FIG. 2B. Comparing SHARP (FIG. 2D) and PCR (FIG. 2B) products at higher template amounts over 6×10⁶ copies, it was noticed that PCR was more prone to forming nonspecific products resulting in a smear, while SHARP produced clean and well-defined amplicons.

SHARP kinetics (FIG. 2C) show an exponential increase in the product amount. The gel image (FIG. 2D) shows one dominant band at 1463 bp corresponding to the desired product. For template copy number below 6×10⁴ and NTC (No Template Control), a small amount of low molecular weight product was observed that was attributed to primer dimers. The primer dimers start accumulating between 20 to 25 minutes, as judged by the kinetics curve of NTC SHARP (FIG. 2C). The primer dimer band was also observed in PCR but is not as pronounced at 30 cycles. It was speculated that ATP hydrolysis by PcrA helicase M6, which is stimulated by single stranded DNA, may deplete ATP in 20-25 minutes at 65° C., shifting the reaction balance toward primer dimer formation when the initial template amount is below the detection limit.

Quantitative or real-time PCR (qPCR) uses and defines the cycle threshold (CT) value as the number of cycles when the fluorescence of a PCR product can be detected above the background. Because the number of amplification cycles in SHARP could not be directly determined, the time it takes to detect the products in qSHARP were instead determined, termed the detection time. The detection threshold was chosen so that the signal for non-template control remains below the threshold for 25 minutes. FIG. 2E displays the initial template copy number versus the measure detection times. From the fit, assuming the exponential model, the doubling time of t½≈1.5 min was determined for the 1464 bp amplicon. For a PCR reaction with Pfusion mix and 1.5 kbp amplicon, each cycle or doubling time is longer than SHARP. It typically takes 2 to 3 minutes for a thermal cycle to melt the duplex, bind primers, replicate, and change the temperature.

SHARP was also demonstrated with other template-primers sets, including ≈3 kbp amplicon (FIG. 3A), and ≈6 kbp amplicon (FIG. 3B). FIG. 3C shows a short 200 bp amplicon using λ-DNA as a template and the kinetics of the amplification reaction. For 200 bp amplicon a gel band was observed even at the single-digit template copy numbers per microliter. For each kinetics curve for 200 bp amplicon (FIG. 3C), the detection time was determined (FIG. 3D) and obtained the doubling time of t_{1/2}=0.83 min or 50 s for the 200 bp product.

FIG. 3E summarizes the SHARP detection limit for different amplicon lengths (200 bp, 1.4 kbp, 3 kbp, and 6 kbp). For short amplicons, under 200 bp, SHARP is extremely sensitive, and it can detect and amplify only a few template molecules per microliter. As the DNA template length increases, the detection limit of SHARP increases. This is also expected with a thermal-cycler based PCR. Other isothermal amplification methods do not achieve amplification of long templates, and they do not report their detection limit (Y. Zhao, et al. “Isothermal Amplification of Nucleic Acids,” Chemical Reviews, vol. 115, p. 12491-12545, 11 2015). FIG. 3F shows the doubling time (t_{1/2}) for 3 different amplicon lengths. The short amplicon has the shortest doubling time of less than 1 minute, while for longer amplicons the time increases. For the 6 kbp amplicon, the low detection limit (FIGS. 3B and 3E) resulted in an insufficient number of kinetics curves to determine the doubling time.

### Applications

Next, it was tested how SHARP behaves with the respect to some common applications in the wet lab. It was first tested whether SHARP makes amplicons that can be propagated in living cells. Starting from a 4012 bp template vector (FIG. 2A), primers were chosen to include the vector backbone containing ampicillin resistance (see Methods), amplified the 3245 bp region with SHARP (FIG. 3A) and ligated the blunt ends. E. coli DH5α cells were transformed with this plasmid and then plated. Three randomly picked colonies were picked, the plasmid extracted, linearized, and the correct 3245 bp product was confirmed in all three (FIG. 4A). Therefore, E. coli can take SHARP-made plasmid, obtain ampicillin resistance, survive, and replicate.

It was further tested whether SHARP could amplify sequences prone to secondary structure formation. A 385-bp region was selected, containing 47 trinucleotide CAG repeats known to undergo multivalent intermolecular interactions in the single stranded form (A. Jain and R. D. Vale, “RNA phase transitions in repeat expansion disorders,” Nature, vol. 546, p. 243-247, 5 2017). Trinucleotide CAG repeats are known to have low PCR yields (L. Aeschbach and V. Dion, “Minimizing carry-over PCR contamination in expanded CAG/CTG repeat instability applications,” Scientific Reports, vol. 7, 12 2017; V. Dion, “Tissue specificity in DNA repair: lessons from trinucleotide repeat instability,” Trends in Genetics, vol. 30, p. 220-229, 6 2014). In the example in FIG. 4B it is shown that a sequence containing (CAG)47 does not inhibit SHARP reaction over a range of SSB concentrations.

Finally, the temperature dependence of the SHARP reaction was tested. With nCOV-2 sequence template and primers targeting 155 bp region, the SHARP reactionwas run in the temperature range between 45° C. and 65° C., and the kinetics curves were recorded (FIG. 4C). The reaction was quenched after 30 minutes and product imaged on 2% agarose gel (FIG. 4D). For the reactions in the range 48.9° C. and 65° C., the 155 bp amplification product was observed, while at lower temperatures amplification did not occur within 30 minutes. At 65° C., it takes about 5 minutes to detect the product, while at 48.9° C. the amplification time increases to 25 minutes (FIG. 4C, inset). Therefore, SHARP with Bst-LF DNAP works optimally at 65° C., but it can also occur at lower temperatures. Bsu and Klenow Exo- are a better choice of DNAP, if SHARP must be carried out at 37° C. FIG. 4E shows 155 bp product amplified at 37° C. with Bsu and Klenow Exo-DNAPs over a range of SSB concentration. The reaction was quenched after 1 hour without producing primer-dimers possibly because PcrA M6 ATPase activity is also lower at 37° C. than at 65° C. It was observed that Klenow Exo-needs higher SSB concentration than Bsu for successful application. SHARP at 37° C. has two limitations, the amplicon length is less than 1 kbp and the amplification time can take more than 1 hour, but lower temperature makes SHARP more versatile when it comes to combining it with other techniques and running in parallel with other temperature-sensitive DNA modifying enzymes.

Overall, it was demonstrated with the examples in FIGS. 2A-2F and 3A-3F that SHARP performs equally well or better than PCR according to the 8 criteria. (1) SHARP can generate up to 6000 bp amplicons in large quantities, unlike existing isothermal amplification methods generating only a few hundred base pair products. (2) SHARP amplification time is dependent on the template and primers, but it usually occurs within 5 to 30 minutes. (3) Primers optimized for PCR can be used with SHARP, unlike many other isothermal methods requiring more complex primers design. (4) SHARP detection limit is comparable to PCR, based on gel assays. (5) SHARP is also suitable for the real-time product monitoring in the presence of intercalating dye, as an isothermal alternative to qPCR. (6) The correct SHARP and PCR product appear as a single band on the gel; hence, it is straightforward to determine the product presence, length, and the amount. Unlike SHARP and PCR, LAMP generates multiple-band product, often containing more than 10 bands, making it challenging to interpret and quantify the result. (7) SHARP product can be used in downstream applications in the same manner as PCR product. For example, it can be gel purified and used for cloning, as was demonstrated in FIGS. 4A-4E. (8) Even when the template is much longer than the amplicon (see the example of lambda DNA template), SHARP works without requiring initial heat denaturing steps.

Engineering PcrA M6 helicase for SHARP: The DNA amplification reaction requires separation of duplex strands and the original patent for PCR (U.S. Pat. No. 4,800,159) proposed separating DNA either through heating or enzymatically with helicases or RecA1. Apart from HAD (M. Vincent, et al., “Helicase-dependent isothermal DNA amplification,” EMBO reports, vol. 5, p. 795-800, 8 2004), which is limited in performance and applications, the strand separation by helicases has not been widely utilized for DNA amplification which it is attributed to the lack of helicases suitable for this application. It was found that PcrA M6 helicase, which was engineered from the Geobacillus stearothermophilus PcrA, serves the purpose well after testing various other candidate helicases.

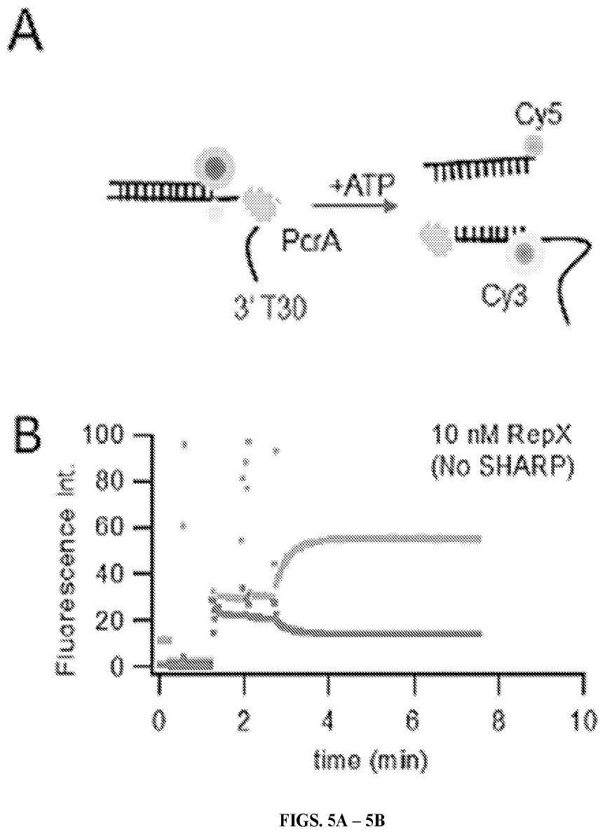

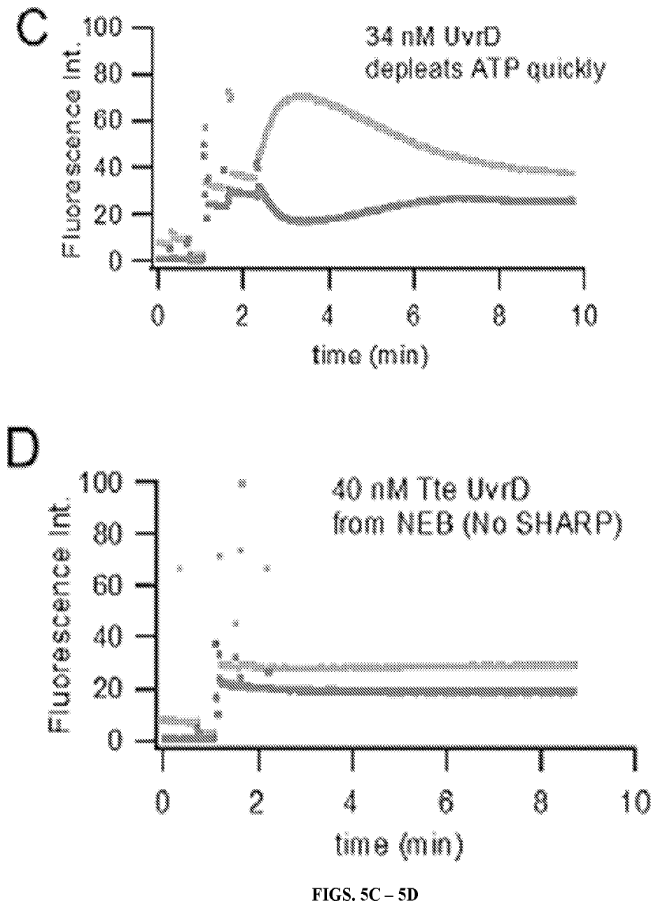

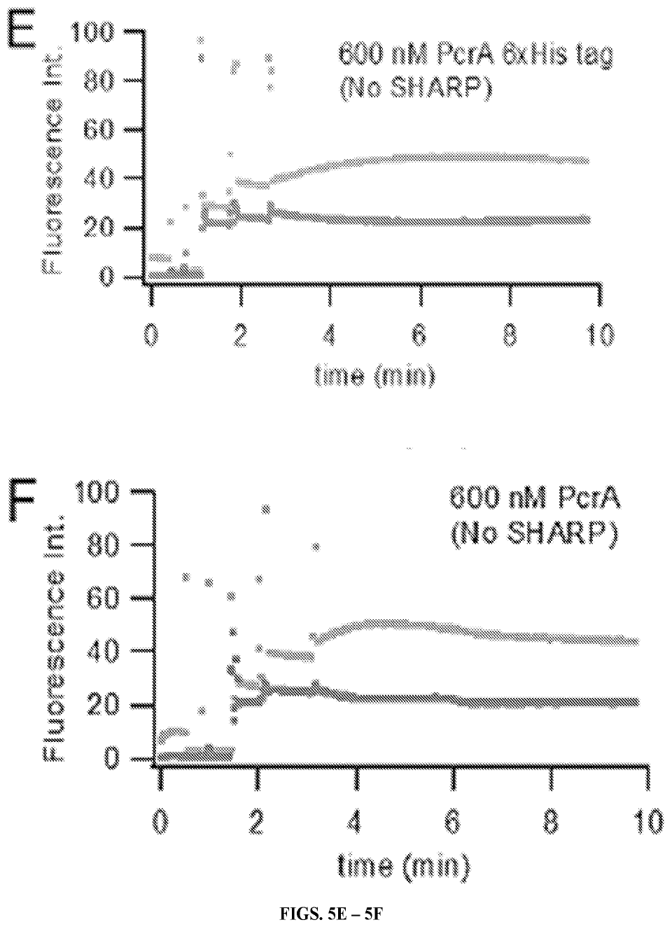

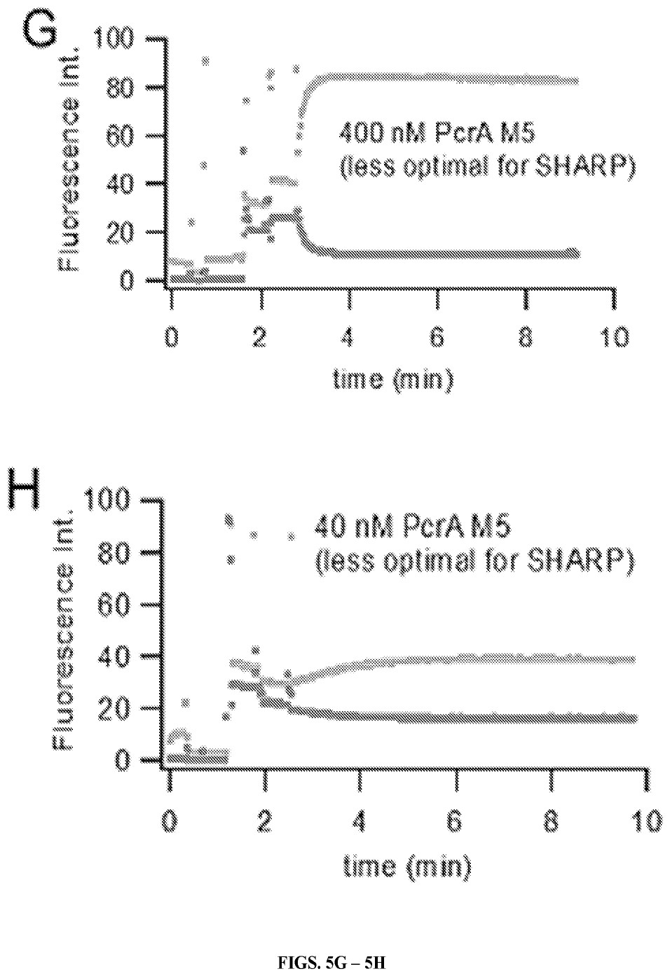

A bulk FRET unwinding assay was used to test the unwinding activity of different helicase candidates for SHARP (S. Arslan, et al., “Engineering of a superhelicase through conformational control,” Science, vol. 348, p. 344-347, 4 2015). The DNA construct (FIG. 5A) consists of 18 bp duplex with 3′ overhang labeled with the FRET pair of Cy3 and Cy5 in such a manner that FRET decreases upon DNA unwinding (FIG. 5A). A reference reaction using a highly-processive engineered Rep-X superhelicase gave rapid FRET decrease upon adding ATP (FIG. 5B). Rep-X showed the highest unwinding activity 37° C., but it is not suitable for SHARP because its activity decreases at elevated temperatures, and it cannot unwind blunt-ended DNA. Rep-X showed no amplification with SHARP. Also tested was the E. coli UvrD helicase for SHARP because UvrD was used with HDA to amplify 100 to 200 bp fragments (M. Vincent et al., “Helicase-dependent isothermal DNA amplification,” EMBO reports, vol. 5, p. 795-800, 8 2004). UvrD at 34 nM is nearly equivalent 10 nM Rep-X when it comes to unwinding the DNA construct (FIG. 5C) but the DNA underwent subsequent reannealing shown as FRET recovery, likely due to ATP depletion. UvrD did not support SHARP. Thermostable Thermoanaerobacter tengcongensis Tte UvrD showed no unwinding activity at 37° C. (FIG. 5D) and no SHARP activity at 65° C.

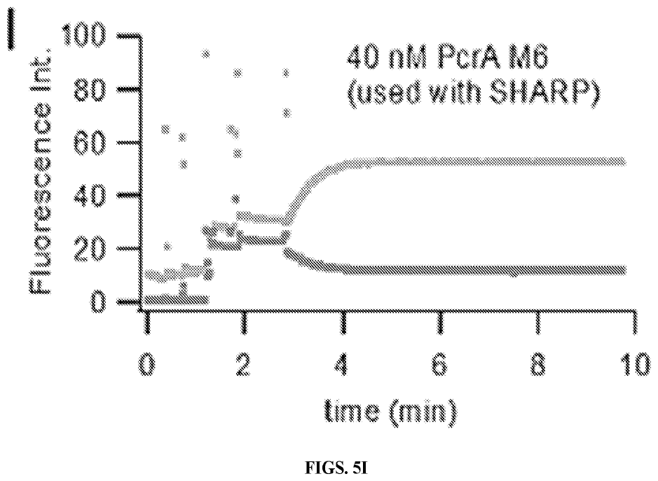

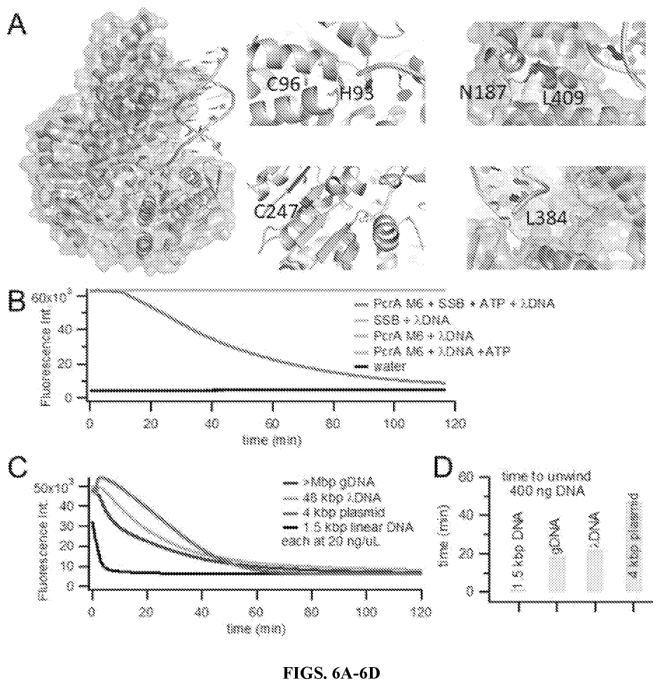

Wild-type Geobacillus stearothermophilus PcrA helicase showed low unwinding activity at elevated concentration of 600 nM (FIGS. 5E and 5F) and did not support SHARP. PcrA M5 mutant, has two native cysteines mutated (C96A and C247A) (FIG. 6A) and two new cysteines introduced (N187C and L409C), and one additional mutation L384V. Engineered PcrA M5 at 400 nM showed unwinding activity (FIG. 5G), which drops significantly at 40 nM (FIG. 5H). PcrA M5 can be also crosslinked to a closed conformation to create a superhelicase, but such crosslinking was not required for SHARP. An additional H93A mutation was introduced in M5 to create PcrA M6. PcrA M6 shows high unwinding activity even at 40 nM (FIG. 5I).

FIG. 6A illustrates locations of 6 point mutations in PcrA M6, mapped to the PcrA structure (S. S. Velankar, et al. “Crystal Structures of Complexes of PcrA DNA Helicase with a DNA Substrate Indicate an Inchworm Mechanism,” vol. 97, p. 75-84, 4 1999. The unwinding activity of PcrA M6 was further characterized on various DNA substrates using intercalating dyes at 37° C. (FIG. 6B). Unwinding was detected as a decrease in the fluorescence intensity over time. PcrA M6 at 40 nM with ATP did not unwind 48 kbp λDNA; however, in the presence of 0.2 mg/mL SSB, almost complete unwinding occurred (FIG. 6B). This ability to unwind long DNA is likely to be important for DNA amplification without an initial heat denaturing step. PcrA M6 at 40 nM concentration could unwind 1.5 kbp linear DNA, long human genomic DNA (gDNA), λ DNA, and 4 kbp circular plasmid in the presence of 0.2 mg/mL SSB and 2 mM ATP (FIG. 6C). For the same mass concentration of DNA substrate used (20 ng/μL), PcrA M6 showed the fastest unwinding for 1.5 kbp linear DNA and also showed unwinding activity for the circular plasmid albeit at the slowest rate among those tested. PcrA plays an important role in plasmid replication and can unwind circular plasmids suggesting that PcrA can unwind DNA without ends. Some of the unwinding characteristics of PcrA M6 and M5 are important for their ability to function in SHARP although more mechanistic studies are needed to pinpoint why these engineered PcrA helicases support SHARP while the wild type PcrA and other related helicases do not.

### Conclusions

A new isothermal amplification method named SHARP was produced with the engineered PcrA M6 helicase. SHARP eliminates thermal cycling from PCR, while keeping all desirable PCR features in place. SHARP can generate simple, linear, and several kilo base pair long amplicons from a template and a set of two primers in under 30 minutes. Apart from replacing thermal cycling from PCR in simple molecular diagnostic, sequencing and gene manipulation reactions such as cloning, SHARP can also detect and amplify sequences prone to forming secondary structures. SHARP has also a potential of being a universal biotechnology tool for many new point-of-care detections methods in medicine, it simplifies many procedures in research labs, and outside labs it enables new applications such as environmental screening, farming pest detection, and many other future consumer-focused diagnostic home tests. The method simply eliminates a bulky and expensive thermal cycler machine from the amplification reaction, thus increasing its versatility.

### References

[1] Y. Zhao, F. Chen, Q. Li, L. Wang and C. Fan, “Isothermal Amplification of Nucleic Acids,” Chemical Reviews, vol. 115, p. 12491-12545, 11 2015.
[2] Y. Zhang and N. A. Tanner, “Isothermal Amplification of Long, Discrete DNA Fragments Facilitated by Single-Stranded Binding Protein,” Scientific Reports, vol. 7, 8 2017.
[3] K. Mullis, F. Faloona, S. Scharf, R. Saiki, G. Horn and H. Erlich, “Specific Enzymatic Amplification of DNA In Vitro: The Polymerase Chain Reaction,” Cold Spring Harbor Symposia on Quantitative Biology, vol. 51, p. 263-273, 1 1986.
[4] K. B. Mullis, H. A. Erlich, N. Arnheim, G. T. Horn, R. K. Saiki and S. J. Scharf, “Process for amplifying, detecting, and/or cloning nucleic acid sequences”. United States of America Patent U.S. Pat. No. 4,800,159A, 24 1 1989.
[5] T. Notomi, “Loop-mediated isothermal amplification of DNA,” Nucleic Acids Research, vol. 28, p. 63e-63, 6 2000.
[6] A. Ganguli, A. Mostafa, J. Berger, M. Y. Aydin, F. Sun, S. A. S. de Ramirez, E. Valera, B. T. Cunningham, W. P. King and R. Bashir, “Rapid isothermal amplification and portable detection system for SARS-COV-2,” Proceedings of the National Academy of Sciences, vol. 117, p. 22727-22735, 8 2020.
[7] O. Piepenburg, C. H. Williams, D. L. Stemple and N. A. Armes, “DNA Detection Using Recombination Proteins,” PLoS Biology, vol. 4, p. e204, 6 2006.
[8] I. Magriñá Lobato and C. O'Sullivan, “Recombinase polymerase amplification: Basics, applications and recent advances,” TrAC Trends in Analytical Chemistry, vol. 98, p. 19-35, 1 2018.
[9] N. Sharma, S. Hoshika, D. Hutter, K. M. Bradley and S. A. Benner, “Recombinase-Based Isothermal Amplification of Nucleic Acids with Self-Avoiding Molecular Recognition Systems (SAMRS),” ChemBioChem, vol. 15, p. 2268-2274, 9 2014.
[10] M. J. Hoser, H. K. Mansukoski, S. W. Morrical and K. E. Eboigbodin, “Strand Invasion Based Amplification (SIBA®): A Novel Isothermal DNA Amplification Technology Demonstrating High Specificity and Sensitivity for a Single Molecule of Target Analyte,” PLoS ONE, vol. 9, p. e112656, 11 2014.
[11] K. E. Eboigbodin and M. J. Hoser, “Multiplex Strand Invasion Based Amplification (mSIBA) assay for detection of Chlamydia trachomatis and Neisseria gonorrhoeae,” Scientific Reports, vol. 6, 2 2016.
[12] M. C. Little, J. Andrews, R. Moore, S. Bustos, L. Jones, C. Embres, G. Durmowicz, J. Harris, D. Berger, K. Yanson, C. Rostkowski, D. Yursis, J. Price, T. Fort, A. Walters, M. Collis, O. Llorin, J. Wood, F. Failing, C. O'Keefe, B. Scrivens, B. Pope, T. Hansen, K. Marino, K. Williams and M. Boenisch, “Strand Displacement Amplification and Homogeneous Real-Time Detection Incorporated in a Second-Generation DNA Probe System, BDProbeTecET,” Clinical Chemistry, vol. 45, p. 777-784, 6 1999.
[13] J. V. Ness, L. K. V. Ness and D. J. Galas, “Isothermal reactions for the amplification of oligonucleotides,” Proceedings of the National Academy of Sciences, vol. 100, p. 4504-4509, 4 2003.
[14] M. Vincent, Y. Xu and H. Kong, “Helicase-dependent isothermal DNA amplification,” EMBO reports, vol. 5, p. 795-800, 8 2004.
[15] A. Jain and R. D. Vale, “RNA phase transitions in repeat expansion disorders,” Nature, vol. 546, p. 243-247, 5 2017.
[16] L. Aeschbach and V. Dion, “Minimizing carry-over PCR contamination in expanded CAG/CTG repeat instability applications,” Scientific Reports, vol. 7, 12 2017.
[17] V. Dion, “Tissue specificity in DNA repair: lessons from trinucleotide repeat instability,” Trends in Genetics, vol. 30, p. 220-229, 6 2014.
[18] S. Arslan, R. Khafizov, C. D. Thomas, Y. R. Chemla and T. Ha, “Engineering of a superhelicase through conformational control,” Science, vol. 348, p. 344-347, 4 2015.
[19] S. S. Velankar, P. Soultanas, M. S. Dillingham, H. S. Subramanya and D. B. Wigley, “Crystal Structures of Complexes of PcrA DNA Helicase with a DNA Substrate Indicate an Inchworm Mechanism,” vol. 97, p. 75-84, 4 1999.
[20] J. Park, S. Myong, A. Niedziela-Majka, K. S. Lee, J. Yu, T. M. Lohman and T. Ha, “PcrA Helicase Dismantles RecA Filaments by Reeling in DNA in Uniform Steps,” Cell, vol. 142, p. 544-555, 8 2010.
[21] T. M. Lohman, J. M. Green and R. S. Beyer, “Large-scale overproduction and rapid purification of the Escherichia coli ssb gene product. Expression of the ssb gene under. lambda. PL control,” Biochemistry, vol. 25, p. 21-25, 1 1986.
[22] S. Bhadra, T. E. Riedel, S. Lakhotia, N. D. Tran and A. D. Ellington, “High-surety isothermal amplification and detection of SARS-COV-2, including with crude enzymes,” 4 2020.
[23] L. An, W. Tang, T. A. Ranalli, H.-J. Kim, J. Wytiaz and H. Kong, “Characterization of a Thermostable UvrD Helicase and Its Participation in Helicase-dependent Amplification,” Journal of Biological Chemistry, vol. 280, p. 28952-28958, 8 2005.
[24] A. F. Slatter, C. D. Thomas and M. R. Webb, “PcrA Helicase Tightly Couples ATP Hydrolysis to Unwinding Double-Stranded DNA, Modulated by the Initiator Protein for Plasmid Replication, RepD,” Biochemistry, vol. 48, p. 6326-6334, 6 2009.
[25] L. T. Chisty, C. P. Toseland, N. Fili, G. I. Mashanov, M. S. Dillingham, J. E. Molloy and M. R. Webb, “Monomeric PcrA helicase processively unwinds plasmid lengths of DNA in the presence of the initiator protein RepD,” vol. 41, p. 5010-5023, 3 2013.

### Example 2

We further show that engineered PcrA M6 is 3 to 4 times faster than prior PcrA M5. FIGS. 5H and 5I in the provisional submission qualitatively compare the unwinding activity of PcrA M5 and M6, respectively. FIG. 5I shows PcrA M6 is more active than M5 in FIG. 5H.

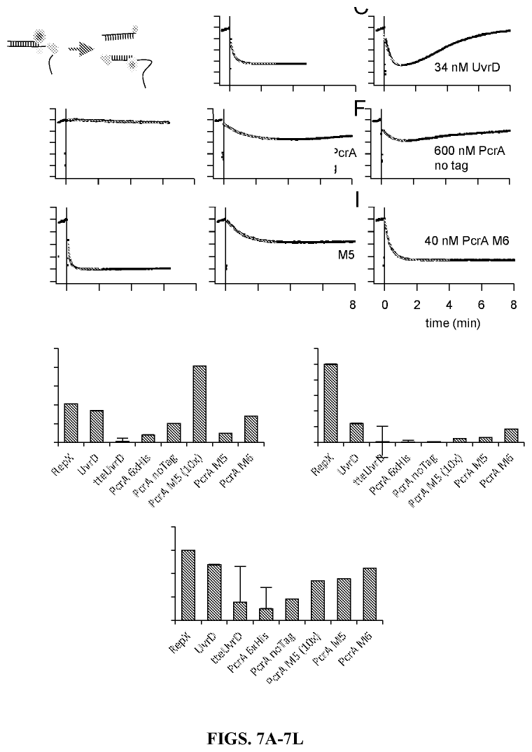

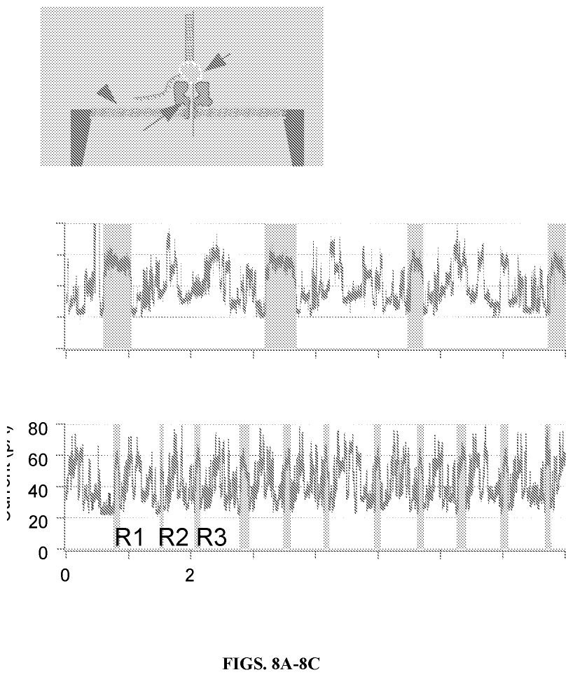

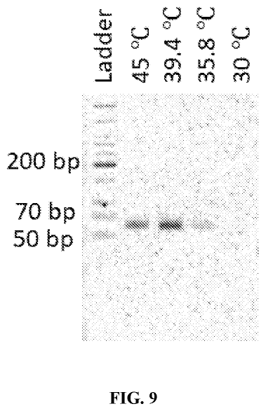

We further analyzed data in FIG. 5 and obtain the unwinding rate for each helicase. FIGS. 7B-71, show extracted FRET efficiencies. From fits, we obtained obtain the unwinding rates and summarized in FIG. 7J. We further divided rates with helicase concentration to obtain the activity, FIG. 7K-L. From obtained normalized activities, we conclude PcrA M6 is 3 times more active than PcrA M5.

We also carried out the nanopore assay, where we directly observe the speed at which a helicase unwinds DNA duplex with Single Molecule Picometer Nanopore Tweezers (SPRNT) in FIG. 8. Briefly, SPRNT setup consists of two separate wells connected with a nanopore. The well near the positive electrode contains the signal buffer (500 mM KCl and 50 mM HEPES pH 8.0), while the well near the negative electrode contains the reaction buffer (200 mM KCl, 50 mM HEPES pH 8.0, 5 mM MgCl2, and 2 mM ATP) and helicase-DNA construct at room temperature (22° C.), FIG. 8A. The DNA construct has 64 repeats of spinach DNA sequence where each repeat is 103 bp longs. We apply 180 mV electric potential difference between the electrodes and measure picoamps (pA) current with Axon Axopatch 200B instrument controlled by the code written in Lab VIEW 2018. As the helicase unwinds the DNA and drives one strand into the nanopore, the electric current passing through the nanopore generates a repetitive signal, FIG. 8B-C. By counting number of 103-bp-long repeats in time, we can determine helicase unwinding speed. For PcrA M5 in FIG. 8B, we observe 3.2 repeats during 8 seconds. For PcrA M6 we identified 10.4 repeats during 8 seconds. From the nanopore data, we extract the unwinding speed of 41 bp/s for PcrA M5 and 134 bp/s for PcrA M6. We conclude that PcrA M6 is more than 3 times fast than PcrA M5.

The higher activity and speed of PcrA M6 demonstrated in both bulk and single-molecule assay facilitates the SHARP amplification.

In the provisional application, we stated that SHARP can amplify RNA targets. We also listed Moloney Murine Leukemia Virus (MMLV) reverse transcriptase; however, we did not include any data to support this claim. To demonstrate SHARP can amplify RNA targets, we used MMLV reverse transcriptase. MMLV first generate cDNA from RNA template, and further acts as a DNA polymerase of moderate efficiency to carry our the amplification. We used nCOV-2 RNA sequence and CDC N2 primers: forward 5′-TTACAAACATTGGCCGCAAA and reverse 5′-GCGCGACATTCCGAAGAA. To generate a 67 bp amplicon at different temperatures. The highest product yield was obtained at 39.4° C. The overall efficiency of RNA amplification is lower than DNA amplification.

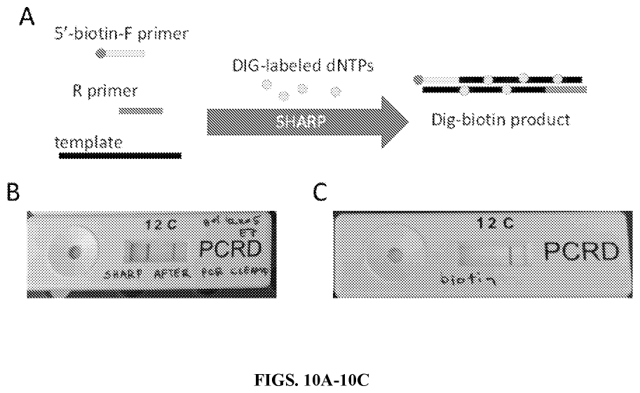

SHARP can be used for portable, point-of-care, point-of-need, and home medical molecular diagnostics tests. SHARP product can be easily detected using a lateral flow device. We carried out SHARP amplification where the forward primer is labeled with biotin and we included DIG-labeled dNTP mix, FIG. 10A. With this modification of the SHARP mix, we can easily pull and detect the SHARP product with the lateral flow device, FIG. 10B. In case no labeled dNTPs are included, the product does not show in the lateral flow device, FIG. 10C

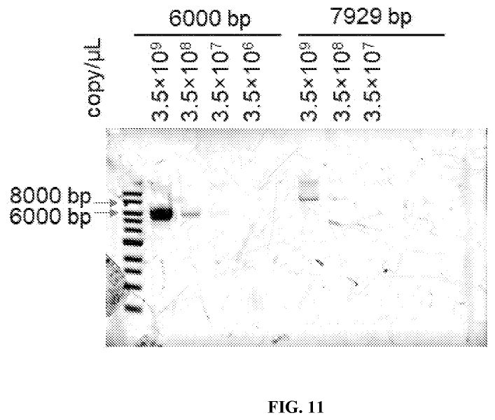

In FIG. 3B, it is shown that that SHARP can generate up to 6000 bp amplicons and showed the 6000 bp amplicon. This is claearly not a limit. SHARP can generate amplicons longer than 6000 base pairs (such as up to or least 6200, 6400, 6600, 6800, 7000, 7200, 7400, 7600, 7800, or 8000 base pairs or more). Yields and detection limits may vary as illustrated in FIG. 3E. FIG. 11 shows previously disclosed 6000 base pairs amplicon and 7929 amplicons obtained with lambda DNA template and forward: 5′-GGCAAAGCGGTGTCCTATAA and reverse 5′-CGGTGTACCTCTCTCGTTTG primers. The correct 7929 product is amplified, but with lower amount than 6000 bp product. Generating amplicons much longer than >8000 base pairs requires extremely high template concentrations and becomes impractical.

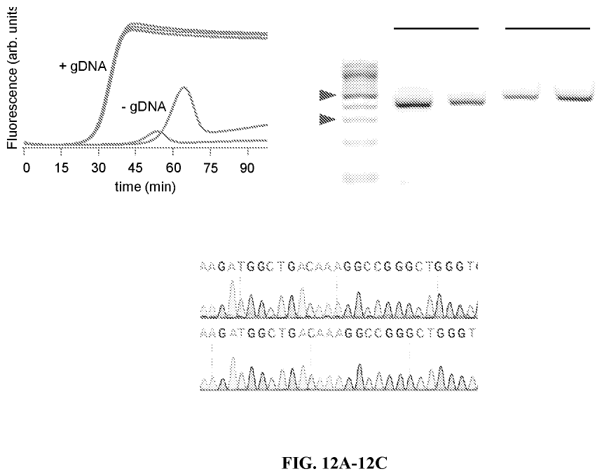

SHARP can amplify regions in genomic DNA from human cells. We purified gDNA from HEK293T cells and amplified a 474 bp DNA region near the DMNT3B gene and another 410 bp region near FANCF gene. We used following primers 5′-AGTTCGCTAATCCCGGAACT (FANCF_F), 5′-AGTTGCCCAGAGTCAAGGAA (FANCF_R), 5′-CCAGTGGTTCAATGGTCATCC (DNMT3B_F), and 5′-GGCCAGTGAAATCACCCTG (DNMT3B_R). FIG. 12 shows the amplification of the product in real time, FIG. 12A, compares it with PCR product in the gel-based assay, FIG. 12B, and confirms the correct product using Sanger sequencing, FIG. 12C.

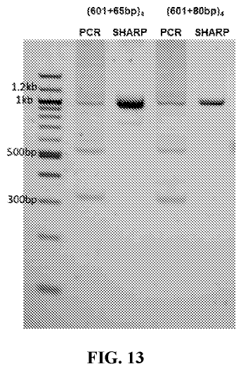

FIG. 4B shows SHARP can amplify repetitive sequence (CAG)n. We have also demonstrated SHARP can amplify 4 repeats of 601 nucleosome positioning DNA sequence and compared SHARP with PCR in FIG. 13. The length of 601 nucleosome positioning sequence is 147 base pair, while linkers between repeats are 65 and 80 base pair long.

### Other Embodiments

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A bacterial derived helicase comprising a PcrA helicase having at least two or more mutations.

2. (canceled)

3. The bacterial derived helicase of claim 1, wherein the PcrA helicase comprises at least a 75% sequence identity to SEQ ID NO: 1.

4. The bacterial derived helicase of claim 1, wherein the PcrA helicase comprises at least a 90% sequence identity to SEQ ID NO: 1.

5-6. (canceled)

7. The bacterial derived helicase of claim 1, wherein the PcrA helicase is derived from Geobacillus.

8. (canceled)

9. A vector encoding a PcrA helicase comprising a 60% sequence identity to SEQ ID NO: 1.

10. (canceled)

11. The vector of claim 9, wherein the PcrA helicase comprises at least a 90% sequence identity to SEQ ID NO: 1.

12-13. (canceled)

14. The vector of claim 9, wherein the PcrA helicase is derived from Geobacillus stearothermophilus.

15-18. (canceled)

19. An engineered helicase comprising a PcrA helicase wherein said PcrA helicase comprises one or more amino acid mutations.

20. The engineered helicase of claim 19, wherein the PcrA helicase is derived from Geobacillus stearothermophilus.

21. The engineered helicase of claim 20, wherein the PcrA helicase comprises mutations at amino positions 93, 96, 187, 247, 384 and 409 of wild type PcrA helicase.

22. A kit comprising an engineered PcrA helicase, a single stranded binding protein (SSB), a Bst polymerase, a thermostable pyrophosphatase (PPase).

23. The kit of claim 22, wherein the engineered PcrA helicase comprises SEQ ID NO: 1.

24-34. (canceled)

35. A method of amplifying nucleic acids comprising: mixing a first composition with one or more primers, a target nucleic acid sequence, and a second composition,
wherein the first composition comprises a nucleic acid dye, deoxyribonucleotide triphosphates (dNTPs), adenosine triphosphate (ATP) or combinations thereof; the second composition comprises: an engineered PcrA helicase, a single stranded binding protein (SSB), a polymerase, a thermostable pyrophosphatase (PPase) buffer or combinations thereof; and the polymerase comprises: DNA polymerase (DNAP) I, DNAP II, DNAP III, DNAP IV, DNAP V. Klenow fragment, reverse transcriptases, Exo-polymerase, high-fidelity polymerases, Taq polymerases, bacteriophage polymerases, Pyrococcus furiosus Vc1 (Pfu) polymerase, Hot Start DNA Polymerase, engineered polymerases, engineered reverse transcriptases or eukaryotic polymerases.

36. The method of claim 35, wherein the mixed compositions are incubated for about 20-60 minutes at a constant temperature in a temperature range of between 37° C. to 90° C.

37-45. (canceled)

46. A method of amplifying nucleic acids comprising: incubating in the absence of thermal cycling i) a composition comprising a target sequence, iii) primers and iii) an engineered PcrA helicase, a single stranded binding protein (SSB), a polymerase, and/or a thermostable pyrophosphatase (PPase) buffer, or combinations thereof.

47. The method of claim 45 wherein the composition is incubated at about 40° C. to about 80° C.

48-50. (canceled)

51. The method of claim 45 wherein the engineered PcrA helicase comprises at least a 90% sequence identity to SEQ ID NO: 1.

52-57. (canceled)

58. A method of screening for cancer comprising the method or kits of claim 22.

59. A method of detecting infectious agents or sepsis comprising the method or kits of claim 22.

60. A method of diagnosing a disease or disorder in a subject comprising the method or kits of claim 22.


SEQUENCE LISTING (11 sequences)

SEQ ID NO: 1 - protein, 724 aa, synthetic construct
MNFLSEQLLAHLNKEQQEAVRTTEGPLLIMAGAGSGKTRVLTHRIAYLMAEKHVAPWNILAITFTNKAAREMRERVQSLLGGAAEDVWISTFASMAVRILRRDIDRIGINRNFSILDPTDQLSVMKTILKEKNIDPKKFEPRTILGTISAAKNELLPPEQFAKRASTYYEKVVSDVYQEYQQRLLRCHSLDFDDLIMTTIQLFDRVPDVLHYYQYKFQYIHIDEYQDTNRAQYTLVKKLAERFQNIAAVGDADQSIYRWRGADIQNILSFERDYPNAKVILLEQNYRSTKRILQAANEVIEHNVNRKPKRLWTENPEGKPILYYEAMNEADEAQFVAGRIREAVERGERRYRDFAVLYRTNAQSRVMEEMLLKANIPYQIVGGVKFYDRKEIKDILAYLRVIANPDDDCSLLRIINVPKRGIGASTIDKLVRYAADHELSLFEALGELEMIGLGAKAAGALAAFRSQLEQWTQLQEYVSVTELVEEVLDKSGYREMLKAERTIEAQSRLENLDEFLSVTKHFENVSDDKSLIAFLTDLALISDLDELNGTEQAAEGDAVMLMTLHAAKGLEFPVVFLIGMEEGIFPHNRSLEDDDEMEEERRLAYVGITRAEEELVLTSAQMRTLFGNIQMNPPSRFLNEIPAHLLETASRRQAGASRPAVSRPQASGAVGSWKVGDRANHRKWGIGTVVSVRGGGDDQELDIAFPSPIGIKRLLAKFAPIEKV

SEQ ID NO: 2 - DNA, 2295 bp, Synthetic construct
  source 1..2295: mol_type = other DNA
catatgcatcaccatcaccatcacgggtccggaatgaactttttatcggaacagctgctcgcccatttaaacaaagagcaacaagaagccgtcaggacgacggaaggcccgctgctcattatggcgggggcgggaagcgggaaaacgcgggtgttgacgcaccgcatcgcctatttgatggcggaaaagcatgtggcgccgtggaacattttggccattacgtttacgaacaaggcggcgcgcgaaatgcgggaacgtgtgcagtcgctcttaggtggggcggcggaagacgtctggatttcgacgttcgcgtcgatggccgtccgcattttgcgccgcgacattgaccgcatcggcatcaaccgcaatttttccatccttgatccgacggaccagctttcagtcatgaaaacgattttaaaagaaaaaaacatagacccgaaaaaatttgagccgcggacgattttaggcacgatcagcgcggcgaaaaacgagctgttgcctccggagcaattcgcgaagcgggcctcgacgtattacgaaaaagtcgtcagcgatgtgtatcaagaataccaacagcgcctgcttcgctgtcattcgctcgattttgacgatttgatcatgacgacgatccaactgtttgaccgcgtgccggatgtgcttcactattaccaatataagtttcagtacattcatattgatgagtaccaggatacgaaccgcgctcaatatacgctggtcaaaaagctggcggaacgctttcaaaacattgccgccgtcggcgacgccgaccaatcgatttatcggtggcgcggggcggacatccaaaacattttgtcgttcgagcgcgactatccgaacgcaaaagtcattttgcttgaacaaaactaccgctcgacgaagcgcattttgcaagcggcgaacgaagtcatcgagcataacgtcaaccggaagccgaaacggctttggacggaaaacccggaaggaaagccgattctttattatgaggcgatgaacgaagcggacgaagcgcagtttgtcgctggacgcatccgcgaggcggtggagcgcggcgaacgccgctaccgtgattttgctgtcttgtaccggacgaacgcccagtcgcgtgtcatggaggaaatgttgctgaaagcgaacattccgtatcaaattgtcggcggcgtaaagttctatgaccggaaagaaattaaagacattctcgcctatttgcgcgtcattgccaatccggacgatgattgcagcttgcttcgcatcattaacgtgccaaaacgcggcattggcgcctcgacgatcgacaaactcgtccgctatgcagccgatcatgagctgtccttgtttgaggcgctcggcgagctagagatgatcgggcttggcgccaaagcggccggggcgctcgccgcgttccgcagccagctcgagcaatggacacagctgcaagaatacgtctccgtcaccgaactcgtcgaagaagtgctcgacaaatcgggctaccgcgagatgctcaaggcggagcggacgattgaagcacaaagccggctcgagaacttggatgagtttttgtcggtgacgaagcattttgaaaatgtgagcgacgataaatcgctcatcgcctttttaaccgacttggcgctcatttccgatttggacgagctgaacgggacggaacaggccgctgaaggagatgccgtcatgttgatgacgttgcatgccgccaaagggctcgagtttccggtcgtctttttgatcggcatggaagaaggcattttcccgcacaaccgctctctcgaggatgacgatgagatggaagaagaacggcggctggcgtacgtcggcatcacccgcgcggaggaagaacttgtgctgacgagcgcgcaaatgcggacgttgtttggcaacatccaaatgaacccgccgtcgcgctttttgaatgaaattccggcgcatttgcttgagacagcctcgcgccgccaagcgggcgcctcccgcccggccgtttcgcgcccgcaggcaagcggcgccgtgggatcgtggaaagtcggcgatcgggcgaatcaccggaaatggggcatcggcaccgtcgtcagcgtccgcggcggcggcgacgaccaagagctcgacatcgccttcccgagcccgatcggcattaaacggttgcttgccaaatttgcgccgattgagaaagtgggtagcggcgactacaaggacgatgacgataaggattataaagacgatgacgataaggactacaaagacgatgacgataaatagggatcc

SEQ ID NO: 3 - protein, 724 aa, Geobacillus stearothermophilus
MNFLSEQLLAHLNKEQQEAVRTTEGPLLIMAGAGSGKTRVLTHRIAYLMAEKHVAPWNILAITFTNKAAREMRERVQSLLGGAAEDVWISTFHSMCVRILRRDIDRIGINRNFSILDPTDQLSVMKTILKEKNIDPKKFEPRTILGTISAAKNELLPPEQFAKRASTYYEKVVSDVYQEYQQRLLRNHSLDFDDLIMTTIQLFDRVPDVLHYYQYKFQYIHIDEYQDTNRAQYTLVKKLAERFQNICAVGDADQSIYRWRGADIQNILSFERDYPNAKVILLEQNYRSTKRILQAANEVIEHNVNRKPKRLWTENPEGKPILYYEAMNEADEAQFVAGRIREAVERGERRYRDFAVLYRTNAQSRVMEEMLLKANIPYQIVGGLKFYDRKEIKDILAYLRVIANPDDDLSLLRIINVPKRGIGASTIDKLVRYAADHELSLFEALGELEMIGLGAKAAGALAAFRSQLEQWTQLQEYVSVTELVEEVLDKSGYREMLKAERTIEAQSRLENLDEFLSVTKHFENVSDDKSLIAFLTDLALISDLDELNGTEQAAEGDAVMLMTLHAAKGLEFPVVFLIGMEEGIFPHNRSLEDDDEMEEERRLAYVGITRAEEELVLTSAQMRTLFGNIQMNPPSRFLNEIPAHLLETASRRQAGASRPAVSRPQASGAVGSWKVGDRANHRKWGIGTVVSVRGGGDDQELDIAFPSPIGIKRLLAKFAPIEKV

SEQ ID NO: 4 - DNA, 23 bp, synthetic construct
  source 1..23: mol_type = other DNA
cccagtcacgacgttgtaaaacg

SEQ ID NO: 5 - DNA, 23 bp, synthetic construct
  source 1..23: mol_type = other DNA
agcggataacaatttcacacagg

SEQ ID NO: 6 - DNA, 19 bp, synthetic construct
  source 1..19: mol_type = other DNA
aattttggggaccaggaac

SEQ ID NO: 7 - DNA, 24 bp, synthetic construct
  source 1..24: mol_type = other DNA
tctggttactgccagttgaatctg

SEQ ID NO: 8 - DNA, 20 bp, synthetic construct
  source 1..20: mol_type = other DNA
cggcttctgactctctttcc

SEQ ID NO: 9 - DNA, 20 bp, synthetic construct
  source 1..20: mol_type = other DNA
ttccttcaagctttgccaca

SEQ ID NO: 10 - DNA, 19 bp, synthetic construct
  source 1..19: mol_type = other DNA
aattttggggaccaggaac

SEQ ID NO: 11 - DNA, 18 bp, synthetic construct
  source 1..18: mol_type = other DNA
gcacctgtgtaggtcaac